SEQUENCE LISTING (6 sequences)

SEQ ID NO: 1 - DNA, 681 bp, mutated hepatitis B virus mHBV
  CDS (1)..(678)
atggagaacatcacatcaggattcctaagacccctgctcgggttacaggcggggtttttcttgttgacaagaatcctcacaataccgcagagtctagactcgtggtggacttctctcagttttctagggggatcacccgtgtgtcttggccaaaattcgcagtccccaacctccaatcactcaccaacctcctgtcctccaacttgtcctggttatcgctggatgtgtctgcggcattttatcatattcctcttcatcctgctgctatgcctcatcttcttattggttcttctggattatcaaggtatgttgcccgtttgtcctcaaactacaagatcaacaacaaccagtacgggatcatgcaaaacctgcacgattcctgctcgcggcaaatctatgtttccctcatgttgctgtacaaaacctacggatggaaattgcacctgtattcccatcccatcgtcttgggctttcgcaagctacctatgggagtgggcctcagtccgtttctcttggctcagtttactagtgccctttgttcagtggttcgtagggctttcccccactgtttggctttcagctatatggatgatgtggtattgggggccaagtctgtacagcatcgtgaggccctttataccgctgttaccaattttcttttgtctctgggtatacatttaa

SEQ ID NO: 2 - protein, 226 aa, mutated hepatitis B virus mHBV
MENITSGFLRPLLGLQAGFFLLTRILTIPQSLDSWWTSLSFLGGSPVCLGQNSQSPTSNHSPTSCPPTCPGYRWMCLRHFIIFLFILLLCLIFLLVLLDYQGMLPVCPQTTRSTTTSTGSCKTCTIPARGKSMFPSCCCTKPTDGNCTCIPIPSSWAFASYLWEWASVRFSWLSLLVPFVQWFVGLSPTVWLSAIWMMWYWGPSLYSIVRPFIPLLPIFFCLWVYI

SEQ ID NO: 3 - DNA, 522 bp, mutated hepatitis B virus mHBV
  CDS (1)..(522)
atgggaggttggtcatcaaaacctcgcaaaggcatggggacgaatctttctgttcccaaccctctgggattctttcccgatcatcagttggaccctgcattcggagccaactcaaacaatccagattgggacttcaaccccatcaaggaccactggccagcagccaaccaggtaggagtgggagcattcgggccagggttcacccctccacacggcggtgttttggggtggagccctcaggctcagggcacattgaccacagtgccaacaattcctcctcctgcatccaccaatcggcagtcaggaaggcagcccactcccatctctccacctctcagagacagtcatcctcaggccatgcagtggaattccactgccttccaccaagctctgcaggatcccagagtcaggggtctgtatcttcctgctggtggctccagttcaggaacagtaaaccctgctccgaatattgcctctcacatctcgtcaatctccgcgaggactggggaccctgtgacgaac

SEQ ID NO: 4 - protein, 174 aa, mutated hepatitis B virus mHBV
MGGWSSKPRKGMGTNLSVPNPLGFFPDHQLDPAFGANSNNPDWDFNPIKDHWPAANQVGVGAFGPGFTPPHGGVLGWSPQAQGTLTTVPTIPPPASTNRQSGRQPTPISPPLRDSHPQAMQWNSTAFHQALQDPRVRGLYLPAGGSSSGTVNPAPNIASHISSISARTGDPVTN

SEQ ID NO: 5 - protein, 226 aa, mutated hepatitis B virus mHBV
MENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSWLSLLVPFVQWFVGLSPTVWLSVIWMMWYWGPSLYNILRPFLPLLPIFFCLWVYI

SEQ ID NO: 6 - protein, 389 aa, mutated hepatitis B virus mHBV
MGQNLSTSNPLGFFPDHQLDPAFRANTANPDWDFNPNKDTWPDANKVGAGAFGLGFTPPHGGLLGWSPQAQGILQTLPANPPPASTNRQSGRQPTPLSPPLGNTHPQAMQWNSTTFHQTLQDPRVRGLYLPAGGSSSGTVNPVPTTVSHISSIFSRIGDPALNMENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSWLSLLVPFVQWFVGLSPTVWLSVIWMMWYWGPSLYNILRPFLPLLPIFFCLWVYI

(19) United States
(12) Patent Application Publication
(10) Pub. No.: US 2003/0129202 A1
(43) Pub. Date: Jul. 10, 2003

(54) Mutated hepatitis b virus, its nucleic and protein constituents and uses thereof

(76) Inventors: Christian Trepo, Bron (FR); Bernard Mandrand, Villeurbanne (FR); Alan Kay, Lyon (FR); Isabelle Chemin, Caluire (FR); Florence Komurian-Pradel, Poleymieux au Mont D'Or (FR)

Correspondence Address: YOUNG & THOMPSON, 745 SOUTH 23RD STREET 2ND FLOOR, ARLINGTON, VA, 22202

(21) Appl. No.: 10/169,668

(22) PCT Filed: Jan. 5, 2001
(86) PCT No.: PCT/FR01/00038

(30) Foreign Application Priority Data
Jan. 6, 2000 (FR) 00/00129

(51) Int. Cl. C12Q 1/70; C07H 21/04; A61K 39/12; C12N 7/00; C12P 21/02; C12N 5/06; C07K 16/08; C12Q 1/68; C12N 15/09; A61K 39/29; C12N 7/01; C12N 15/00; C12N 15/63; C12N 15/70; C12N 15/74; C12N 5/00; C12N 5/02; C07K 16/00; C12P 21/08

(52) U.S. Cl. 424/225.1; 435/5; 435/69.3; 435/235.1; 435/320.1; 435/325; 530/388.3; 424/186.1; 536/23.72; 435/6

(57) ABSTRACT

The invention concerns an isolated mHBV having the following characteristics: (i) a genome with partly double-strand circular DNA, (ii) the genome including the Pre-S, S, C, P and X genes, (iii) the Pre-S genes coding for surface antigens, the S gene coding for a HBsAg envelope protein, the C gene coding for a HBeAg protein and aHBcAg protein, the P gene coding for a DNA reverse polymerase/transcriptase enzyme and the X gene coding for a HBxAg protein. The invention is characterised in that the gene S comprises a DNA nucleotide sequence referenced SEQ ID NO 1 and the Pre-S gene comprises a nucleotide sequence referenced SED ID NO 3. The invention also concerns DNA molecule, RNA molecule, modified surface proteins and their uses in particular for diagnostic, therapeutic and vaccine purposes.

Five types of viral hepatitis—hepatitis A, B, C, D, E—are now quite well known. In each case the virus invades the liver and provokes an inflammatory state with destruction of the hepatic cells.

Hepatitis B is caused by a virus, the human hepatitis B virus (HBV). The HBV virus was discovered by Blumberg et al.: A “new” antigen in leukemia sera, JAMA 191: 541, (1965). The virus is transmitted in the blood, by sexual contact or by perinatal transmission.

In most cases infection by HBV does not lead to any symptoms and is responsible for asymptomatic acute hepatitis. Acute hepatitis is characterized by digestive disorders, abdominal pains, coloration of the urine and abnormal, discoloured faeces, asthenia and jaundice. Acute hepatitis can develop into a fulminant form with rapid liver necrosis.

The viral infection can also develop into a chronic form, either in patients who have exhibited acute hepatitis, or in individuals for whom the infection was asymptomatic. Chronic carriers exhibit hepatic lesions of varying severity and an increased risk of developing cirrhosis and primitive liver cancer. In Asia and Africa, where infections are often chronic, primitive liver cancers represent a crucial public health problem. In addition, chronic carriers are reservoirs for the virus and permit it to spread, transposing the public health problem to a global problem.

Infection with HBV is one of the commonest viral infections in man. It is a disease of wide occurrence with a distinct geographic incidence. In Europe and North America between about 0.1% and 1% of the population is infected, whereas in Asia and Africa up to 20% of the population are HBV carriers. It is estimated that about 350 million people are infected with HBV throughout the world. Hierarchical organization of viral infections following a transfusion shows that HBV is transmitted first, followed by HCV and then HIV. HBV is a small DNA virus with a diameter of 42 nm, which belongs to the group of hepatotropic DNA viruses (hepadnaviruses) and is classified in the Hepadnaviridae family. Its genomic structure is remarkably compact. The virus comprises an outer envelope and a nucleocapsid. The envelope is composed principally of three surface antigens (HBsAgs: hepatitis B surface antigens) which play a major role in the diagnosis of HBV infections. The nucleocapsid contains the core antigen (HBcAg), a DNA polymerase/reverse transcriptase, as well as the viral genome. The viral core constitutes the infectious element of the virus and the outer membrane carries the main antigenic determinant (epitope) of the virus, the HBs antigen. The viral core remains inside the nucleocapsid. It is about 28 nm in diameter.

Despite its small size (3200 base pairs), the circular, partially double-stranded DNA of HBV codes for four types of viral products starting from its overlapping genes S, C, P, and X.

The S gene codes for the HBsAg envelope protein expressed on the external surface of the virion. The HBsAg envelope protein is made up of two main polypeptides, a 24 kDa polypeptide and its 28 kDa glycosylated form. A certain number of subdeterminants of HBsAg have been identified. Subdeterminant a is carried by all the HBsAg isolates. However, HBsAg can additionally contain a specific antigen of the subtypes d or y, w or r. Upstream from the S gene, the Pre-S genes code for various HBV surface antigens.

The P gene codes for DNA polymerase/reverse transcriptase, which is very important in the mechanism of viral replication.

The C gene codes for two proteins of the nucleocapsid: HBeAg which is a secreted soluble protein, and HBcAg, the intracellular core protein. HBeAg is a serological marker of increased viral replication.

The X gene codes for HBxAg, which has various biological effects and in particular can transactivate the transcription of viral and cellular genes.

When HBV infects an individual, the viral DNA replicates entirely within the hepatic cells of the host.

After infection with HBV, the first marker that can be detected in the patient's serum is the HBsAg antigen, but this marker rarely persists beyond six months. After the HBs antigen has disappeared from the serum, the anti-HBsAg antibodies become detectable and persist. Because the HBc antigen is sequestered by the HBs envelope antigen, it is not routinely detectable in patients' serum, but the presence of anti-HBc antibodies can easily be demonstrated in the first or second weeks following appearance of the HBs antigen.

It is now certain, however, that the conventional serological tests, employing the aforementioned markers, do not permit the variants of HBV to be detected. The fact that patients who are carriers of HBV and have developed chronic hepatitis B exist, without it being possible to demonstrate HBV infection using the conventional serological markers, is of the utmost importance and shows that better tests need to be developed, especially in the context of organ transplantation and for the treatment of patients.

The existence of HBV variants has been suspected for many years. This assumption is based on the detection of viral DNA in the serum and/or the liver of patients with chronic hepatitis, in the absence of detection of the conventional serological markers (HBsAg and anti-HBc).

The inability to detect HBsAg in patients who are carriers of DNA sequences of the virus might have several explanations, such as poor expression of the surface antigen or the presence of mutations at the level of the antigenic determinant of the S protein. In the first case, a viral coinfection might suppress HBV replication (Jilg W. et al., J. Hepatol., 1995, 23: 14-20, Jylberberg et al., Clinical infection diseases, 1996, 23: 1117-1125, Ushida et al., J. of Med. Virol., 1997, 52: 399-405, Hofer et al., Eur. J. Clin.; Microbiol. Infect. Dis., 1998, 17: 6-13. Another explanation might be that the HBs antigen is masked during the formation in vivo of immune complexes with the anti-HBs antibodies.

The present inventors have now identified and characterized a new variant or mutant of hepatitis B, whose detection eludes certain commercial serological tests using a polyclonal antibody both in capture and in detection. The new variant or mutant has, among others, mutations at the level of the gene coding for the HBs antigen. For the purpose of the present patent application, they have called this new variant or mutant mHBV.

In addition the present inventors have shown that the negativity of the commercially available tests might be due to these mutations at the level of the subdeterminant a of the HBs antigen. In fact, the subdeterminant a is a major antigenic site of the surface antigen of HBV and mutations at this level explain the absence of detection by the existing tests.

Thus, the present invention relates to the mHBV virus, whose genomic DNA includes a nucleotide sequence of the S gene that codes for an HBsm antigen (mutated HBs antigen), the said nucleotide sequence being referenced SEQ ID NO 1. The genomic DNA of mHBV also includes a nucleotide sequence of the Pre-S gene referenced SEQ ID NO 3.

The mHBV virus has the following characteristics:

(i) a genome with partially double-stranded, circular DNA,

(ii) the said genome containing the genes Pre-S, S, C, P and X,

(iii) the Pre-S gene coding for surface antigens, the S gene coding for an HBsAg envelope protein, the C gene coding for an HBeAg protein and an HBcAg protein, the P gene coding for a DNA polymerase/reverse transcriptase enzyme and the X gene coding for an HBxAg protein, and is characterized in that the S gene includes a DNA nucleotide sequence with the reference SEQ ID NO 1 and in that the Pre-S region includes a DNA nucleotide sequence with the reference SEQ ID NO 3; it being understood that the remainder of the genome of the mHBV virus is roughly identical to the genome of the wild-type HBV virus, as has been demonstrated by the inventors by complete sequencing of the genome of the mHBV virus.

The invention also relates to a DNA molecule, characterized in that it includes a DNA nucleotide sequence selected from SEQ ID NO 1, SEQ ID NO 3, their fragments as defined below, and their complementary sequences and an RNA molecule, characterized in that it includes an RNA nucleotide sequence that is the product of transcription of a DNA nucleotide sequence selected from SEQ ID NO 1, SEQ ID NO 3, their fragments and their complementary sequences.

The invention also relates to a modified surface protein, characterized in that it includes or consists of a peptide sequence selected from SEQ ID NO 2, SEQ ID NO 4 and their fragments as defined below.

The invention also relates to a DNA or RNA nucleotide fragment of at least 12 nucleotides, preferably of at least 15 nucleotides or 18 nucleotides and advantageously of at least 21 nucleotides and that includes a DNA nucleotide sequence that includes the nucleotides 325 to 336 of SEQ ID NO 1 and/or the nucleotides 235 to 237 of SEQ ID NO 1 and/or the nucleotides 391 to 393 of SEQ ID NO 1 and/or the nucleotides 478 to 480 of SEQ ID NO 1 and/or the nucleotides 28 to 30 of SEQ ID NO 1 and/or the nucleotides 39 to 41 of SEQ ID NO 1 and/or the nucleotides 358 to 360 of SEQ ID NO 1 and/or the nucleotides 385 to 387 of SEQ ID NO 1 and/or the nucleotides 118 to 120 of SEQ ID NO 1 and/or the nucleotides 628 to 630 of SEQ ID NO 1 and/or the nucleotides 249 to 251 of SEQ ID NO 3, and/or the nucleotides 250 to 252 of SEQ ID NO 3, or is the product of transcription of the said DNA nucleotide sequences; a DNA or RNA nucleotide fragment that includes a nucleotide sequence that includes the DNA nucleotide sequences SEQ ID NO 1 and SEQ ID NO 3 or the complementary sequences of the said sequences SEQ ID NO 1 and SEQ ID NO 2 or the RNA nucleotide sequences that are the products of transcription of sequences SEQ ID NO 1 and SEQ ID NO 3; and a DNA or RNA nucleotide fragment, characterized in that it consists of a DNA nucleotide sequence that corresponds to SEQ ID NO 1 and SEQ ID NO 3 or the complementary sequences of the said sequences SEQ ID NO 1 and SEQ ID NO 3 or in that it consists of an RNA nucleotide sequence that corresponds to the products of transcription of sequences SEQ ID NO 1 and SEQ ID NO 3.

Advantageously, the aforementioned fragments containing the nucleotides 250 to 252 of SEQ ID NO 3 are fragments containing at least 21 nucleotides.

More advantageously, when the aforementioned fragments include the nucleotides 628 to 630 of SEQ ID NO 1, the said fragments also include the nucleotides 325 to 336, and/or 235 to 237, and/or 391 to 393, and/or 478 to 480, and/or 28 to 30, and/or 39 to 41, and/or 358 to 360, and/or 385 to 387, and/or 118 to 120 of SEQ ID NO 1, and/or the nucleotides 250 to 252 of SEQ ID NO 3.

The invention further relates to a protein fragment, characterized in that it includes a peptide sequence of at least 4 amino acids, preferably of at least 5 or 6 amino acids and advantageously of at least 7 amino acids, especially of 6 to 15 amino acids and advantageously of 6 to 10 or of 8 to 12 amino acids, and which includes the amino acids 109-112 and/or 79 and/or 131 and/or 160 and/or 10 and/or 14 and/or 120 and/or 129 and/or 40 and/or 210 of SEQ ID NO 2 and/or the amino acid 84 of SEQ ID NO 4; a protein fragment that includes or consists of a peptide sequence that includes the peptide sequences SEQ ID NO 2 and SEQ ID NO 4; a protein fragment whose peptide sequence consists of a sequence selected from SEQ ID NO 2 and SEQ ID NO 4.

Advantageously, the aforementioned fragments containing the amino acid 84 of SEQ ID NO 4 are fragments containing at least 7 amino acids.

More advantageously, when the aforementioned fragments include the amino acid nucleotides 210 of SEQ ID NO 2, the said fragments also include the amino acids 109-112 and/or 79 and/or 131 and/or 160 and/or 10 and/or 14 and/or 120 and/or 129 and/or 40 of SEQ ID NO 2 and/or the amino acid 84 of SEQ ID NO 4.

The mutated HBsAg protein has antigenic and/or immunologic characteristics that are different from the wild-type HBsAg protein and in particular is not recognized by polyclonal antibodies directed against the wild-type protein.

The mutated protein (HBsAgm) and/or the mutated surface protein Pre-S (Pre-Sm) can be obtained by peptide synthesis or by techniques of genetic recombination that are well known to a person skilled in the art.

The methods of construction, manipulation and verification of recombinant DNA molecules and of nucleotide sequences are well known to a person skilled in the art. To modify the gene that codes for the HBsAgm protein and/or the Pre-Sm protein and obtain the HBsAgm and/or Pre-Sm proteins of the invention, it is necessary to insert the codons CAA, ACT, ACA, AGA, CAT, AAA, AGC, AGA, GGG, CGC, AGT, AGG or any other codon that codes for the amino acids Gln, Thr, Thr, Arg, His, Lys, Ser, Arg, Gly, Arg, Ser, Arg respectively in positions 109, 110, 111, 112, 79, 131, 160, 10, 14, 129, 40 and 210 of SEQ ID NO 2 and/or the codon ACA that codes for Thr or any other codon that codes for this amino acid in position 84 of SEQ ID NO 4.

The significant mutations of mHBV have been identified and demonstrated by cloning, sequencing and alignment of the nucleotide and protein sequences of mHBV relative to the 102 sequences of the HBs antigen of the NBRF/PIR base, available on the Infobiogen server of Villejuif.

Several methods are available for carrying out the appropriate sequence modifications. One suitable method is synthesis de novo, by phosphoramidite or phosphite chemistry, of the desired sequence using the frequencies of virus or yeast codons. DNA synthesis can be effected starting from commercial elements. An example of the said DNA synthesis is described by Hayden and Mandecki, DNA 7: 571 (1988). Another method is cloning, in a suitable single-stranded vector, of a suitable restriction fragment starting from a vector that already contains the HBV genome and then carrying out a directed mutagenesis in vitro, as described for example by Bolstein et al., Science, 229, 1193 (1982). A culture of E. coli K12, strain C600 containing the recombinant plasmid pRIT10601 containing an HBV genome of subtype ay cloned in pBR322 was deposited at the ATCC on Jun. 2, 1982 under accession number ATCC 39132, in accordance with the provisions of the Budapest Treaty. The sequence of the S gene coding for the HBsAgm protein or longer sequences also coding for the polypeptides Pre-S can be excised from the said clones by conventional techniques. A suitable restriction fragment is for example the fragment Xbal-Accl of the coding region of the S gene of pRIT10601. Vectorization systems for in vitro mutagenesis are available commercially. The mutated gene fragment is then reintroduced into the S gene. Another method of obtaining the required sequence modifications is the use of PCR (Polymerase Chain Reaction) as described by Ho et al., Gene, 77, 51 (1989). In each case the coding sequence for a mutated protein is expressed in a suitable host cell under the control of a suitable promoter. It is thus possible to use a functional expression cassette in a cell from a eukaryotic or prokaryotic organism permitting expression of the S gene coding for the HBsAgm protein and/or expression of the mutated surface protein encoded by the Pre-S gene or expression of fragments of these proteins, the gene being placed under the control of the elements necessary for its expression. Among the microbial systems, Escherichia coli and Saccharomyces cerevisiae have been widely used for the expression of recombinant proteins, but expression of the HBs antigen in a prokaryotic system, such as E. coli, has proved to be very difficult. Preferably, the cell is a cell obtained from a eukaryotic organism, such as the CHO or COS cells and advantageously a cell obtained from a lower eukaryotic organism, such as yeast cells. The HBsAgm recombinant protein can be obtained in a cell of Saccharomyces cerevisiae as described by Harford et al., Develop. Biol. Standard. 54: page 125 (1983), Valenzuela et al., Nature 298, page 347 (1982) and Bitter et al., J. Med. Virol. 25, page 123 (1988) or expressed in Pichia pastoris, as described by Gregg et al., Biotechnology, 5, page 479 (1987). The surface proteins of mHBV can also be expressed in a mutant strain of Saccharomyces cerevisiae as described by Kniskern et al. in U.S. Pat. No. 5,614,384.

Thus, the present invention also encompasses a functional expression cassette in a cell originating from a prokaryotic or eukaryotic organism permitting the expression of a DNA sequence or of a DNA fragment as defined previously, placed under the control of the elements necessary for its expression; the vector containing the expression cassette and the cell obtained from a prokaryotic or eukaryotic organism, preferably a lower eukaryotic organism and advantageously a cell obtained from Saccharomyces cerevisiae or from Pichia pastoris containing the expression cassette or the vector, as well as the surface protein produced by the expression cassette, the vector or the cell.

The method for preparing a modified recombinant surface protein of the invention consists of culturing a host cell as defined above in a suitable culture medium, the said host cell being transformed with an expression vector that contains a DNA nucleotide sequence such as represented in SEQ ID NO 1 and/or SEQ ID NO 3, their fragments and their complementary sequences or a nucleotide fragment as defined previously, and purifying the said modified surface protein produced to a required degree of purity.

Another object of the invention is an immunogenic peptide that has a peptide sequence as defined previously and that consists of a recombinant protein obtained according to the aforementioned protocols and its use for the production of a monoclonal or polyclonal antibody by immunization of a mammal, preferably a mouse, a rat or a rabbit, with the said immunogenic peptide. The production of polyclonal and monoclonal antibodies forms part of the general knowledge of a person skilled in the art. As a reference we may mention Köhler G. and Milstein C. (1975): Continuous culture of fused cells secreting antibody of predefined specificity, Nature 256: 495-497 and Galfre G. et al. (1977) Nature, 266: 522-550 for the production of monoclonal antibodies and Roda A., Bolelli G. F.: Production of high-titer antibody to bile acids, Journal of Steroid Biochemistry, Vol. 13, pp. 449-454 (1980) for the production of polyclonal antibodies. Antibodies can also be produced by immunization of mice or of rabbits with the viral particles of mHBV. For the production of monoclonal antibodies, the immunogen can be coupled to keyhole-limpet haemocyanin (KLH peptide) as immunization substrate or to serum albumin (SA peptide). The animals are injected with immunogen using Freund complete adjuvant. The sera and supernatants from hybridoma cultures from the immunized animals are analysed for their specificity and selectivity using conventional techniques, for example ELISA or Western Blot tests. The hybridomas producing the most specific and most sensitive antibodies are selected. Monoclonal antibodies can also be produced in vitro by cellular culture of the hybridomas produced or by recovery of ascites fluid, after intraperitoneal injection of the hybridomas in mice. Regardless of the manner of production, in supernatant or in ascites, the antibodies are then purified. The methods of purification used are essentially filtration on ion-exchanger gel and exclusion chromatography or affinity chromatography (protein A or G). A sufficient number of antibodies are screened in functional tests to identify the antibodies with best performance. The production in vitro of antibodies, of antibody fragments or of antibody derivatives, such as chimaeric antibodies produced by genetic engineering, is well known to a person skilled in the art.

More particularly, by antibody fragment we mean the fragments F(ab)2, Fab; Fab′, sFv (Blazar et al., 1997, Journal of Immunology 159: 5821-5833 and Bird et al., 1988, Science 242: 423-426) of a native antibody, and by derivative we mean, inter alia, a chimaeric derivative of a native antibody (see for example Arakawa et al., 1996, J. Biochem. 120: 657-662 and Chaudray et al., 1989, Nature 339: 394-397).

The monoclonal or polyclonal antibody thus obtained is incorporated in a diagnostic composition that is used in a method for detecting at least one mutated surface protein that consists of SEQ ID NO 2 and/or SEQ ID NO 4 in a biological sample, according to which the biological sample is placed in contact with the said diagnostic composition in predetermined conditions permitting antibody/antigen complexes to form, and the formation of the said complexes is detected. In particular, the monoclonal antibodies obtained are specific to the required mutated protein and do not recognize the wild-type protein, for example a wild-type HBsAg protein.

The invention also relates to a diagnostic composition for the detection of auto-antibodies in a biological sample, the said composition containing, inter alia, a protein or a mutated protein fragment as previously defined and the method for detecting the said auto-antibodies directed against at least one mutated surface protein consisting of SEQ ID NO 2 and/or SEQ ID NO 4 in a biological sample, according to which the biological sample is brought into contact with the diagnostic composition in predetermined conditions permitting antibody/antigen complexes to form, and the formation of the said complexes is detected.

Cloning of the genome of virions of hepatitis B of various serotypes is well known. As a reference we may cite, among others: Miller et al., Hepatology, 9 (1989), page 322.

The present invention also relates to a vaccine against the mHBV virus. This vaccine is prepared according to the known methods already used for the preparation of commercially available vaccines. This vaccine contains at least the protein HBsAgm and/or the protein Pre-Sm, either in native form, or in recombinant form, or a synthetic polypeptide whose peptide sequence corresponds to the amino acid sequence of HBsAgm and/or Pre-Sm, or fragments of the said proteins and of the said polypeptide. The HBsAgm and/or Pre-Sm proteins in native form are recovered from the plasma of patients infected with mHBV. The HBsAgm and/or Pre-Sm proteins in recombinant form are obtained by using a functional expression cassette in a cell originating from a eukaryotic or prokaryotic organism permitting expression of the S gene coding for the HBsAgm protein and/or of the Pre-Sm gene coding for the Pre-Sm region, placed under the control of the elements necessary for its expression. Preferably, the cell is a cell obtained from a eukaryotic organism, such as yeast cells. The HBsAgm and/or Pre-Sm recombinant proteins for the production of vaccines can be obtained in a cell of Saccharomyces cerevisiae as described by Harford et al., Develop. Biol. Standard. 54: page 125 (1983), Valenzuela et al., Nature 298, page 347 (1982) and Bitter et al., J. Med. Virol. 25, page 123 (1988) or expressed in Pichia pastoris, as described by Gregg et al., Biotechnology, 5, page 479 (1987). The vaccines can also be prepared starting from hybrid immunogenic particles containing the HBsAgm protein and/or the Pre-Sm protein, as described in patent application EP 0 278 940. The said particles can contain, for example, all or part of the precursor protein of HBsAgm encoded by the coding sequence immediately preceding the S gene in the HBV genome, i.e. the Pre-S coding sequence. The vaccine can additionally contain the Pre-Sm protein of the invention, either isolated and purified from patients' plasma, or obtained by genetic recombination, or obtained by peptide synthesis or a fragment of the said protein. Advantageously, the vaccine contains the proteins HBsAgm and Pre-Sm defined previously, optionally combined with the proteins HBsAgm′ and/or Pre-Sm′, defined later, or with their fragments and/or with the proteins HBsAg and/or Pre-S or their fragments, of wild type; it being understood that the proteins HBsAgm′, Pre-Sm′, HBsAg and Pre-S comply with the general definitions given for the proteins HBsAgm and Pre-Sm.

An immunogenic or vaccinal composition according to the invention is a composition that contains a protein or a protein fragment as defined above, optionally combined with a vehicle and/or a suitable adjuvant and/or a pharmaceutically acceptable excipient. The vaccines containing the HBsAgm protein and/or the Pre-Sm protein or their fragments are prepared conventionally and contain an immunoprotective quantity of the HBsAgm protein and/or of the Pre-Sm protein and/or of their fragments, preferably in a buffered saline solution and mixed or adsorbed by means of known adjuvants, such as aluminium hydroxide and phosphate.

The present invention also relates to vaccines including nucleic acid molecules that code for one or more protein(s) of the invention or for immunogenic peptides or their fragment(s). The nucleic acid vaccines, especially the DNA vaccines, are generally administered in combination with a pharmaceutically acceptable vehicle by intramuscular or subcutaneous injection. The aforementioned nucleic acid vaccines may additionally contain nucleic acid molecules that code for the proteins HBsAgm′ and/or Pre-Sm′ and/or HBsAg and/or Pre-S defined above. These vaccines are composed of at least one gene coding for at least one protein or antigen of the invention whose expression is controlled by a strong promoter, preferably a mammalian promoter, expressed on a DNA plasmid or vector of bacterial origin. When administered by intramuscular or subcutaneous injection, the DNA vaccines are transcribed and translated and the protein that they encode is presented to the immune system, inducing a humoral and cellular response. One of the main advantages of the DNA vaccines is that they can be constructed and manipulated. They are able to supply their own adjuvant in the form of CpG sequences present in the bacterial DNA. The DNA vaccines provoke the de novo synthesis of proteins in the transfected cells, leading to combination of antigenic peptides with the determinants of MHC I and hence activation of cytotoxic T cells. Furthermore, DNA vaccines do not induce measurable immune responses on the vector or plasmid, thus permitting repeated use.

The term “immunoprotective” signifies that a sufficient quantity of protein, especially of HBsAgm protein and/or of Pre-Sm protein or of their fragments, is administered to an individual to induce antibody production (humoral immune response) sufficient for it to be protective or an immune response mediated by the cytotoxic cells (cellular immune response) to confer protection against the infectious agent without producing side effects. The two types of response differ in that the antibodies recognize the antigens in their three-dimensional form whereas the cytotoxic cells recognize portions of the said antigens, associated with glycoproteins encoded by the major histocompatibility complex (MHC). The cytotoxic T lymphocytes (CTLs) play an essential role in the defence of virus-infected cells. They act directly by cytotoxicity but also by supplying specific and non-specific aid to other immunocytes, such as macrophages, B cells and the other T cells. The infected cells transform the antigen through intracellular events involving proteases. The transformed antigen is then presented to the surface of the cells in the form of peptides bound to HLA class I molecules at the level of the T cell receptors on the CTLs. The class I MHC molecules can also bind exogenous peptides and present them to the CTLs without intracellular transformation. Chisari et al. (Microbiol. Pathogen, 6: 31 (1989)) suggested that hepatic lesions could be mediated by a response of the CD8+ cytotoxic T cells restricted by the HLA class I to the antigens encoded by the HBV. The commercially available vaccines against HBV, which use either the HBsAg protein purified from the plasma of HBV carriers at a chronic stage of the disease, or a recombinant HBsAg protein, or synthetic peptides, only endow a person with real protection in about 90% of cases. In consequence, persons who are not immunized, or are immunized but not protected, constitute a significant reservoir for potential infection. It is therefore important to stimulate the cellular immune response of the individuals to obtain an appropriate response to the HBV antigens. Moriyama et al., Science, 248: 361-364 (1990) reported that the major envelope antigen of HBV (HBsAg) is expressed on the surface of hepatocytes in a form that can be recognized by specific antibodies of the envelope and by the CD8+ cytotoxic T lymphocytes restricted by the class I MHC molecules.

Thus, the present invention also relates to peptides that induce responses of cytotoxic T lymphocytes restricted by the class I MHC molecules, derived from SEQ ID NO 2, whose peptide sequence consists of a sequence of at least 6 amino acids, preferably of at least 8 or 9 amino acids and advantageously of 8 to 12 contiguous amino acids, the said sequence being selected from SEQ ID NO 2 and inducing a response of the cytotoxic T lymphocytes restricted by the class 1 MHC molecules and their uses in an immunogenic composition.

The quantity of protein or peptide administered depends on whether or not an adjuvant is added, but is generally between 10 and 50 μg/ml of protein or peptide. Thus, commonly, it is administered in a dose of 20 μg/0.5 ml of protein in adults and 10 μg/0.5 ml in children. The HBsAgm protein and/or the Pre-Sm protein and/or their fragments can also be mixed with the HBsAg and/or Pre-S proteins or fragments of the said proteins of wild type for the formulation of a vaccine. They can also be mixed with hybrid particles bearing epitopes of proteins of other organisms or with other immunogenic compounds for the formulation of bivalent or polyvalent vaccines. The preparation of vaccines is described in particular in “Vaccines”, ed. Voller et al., University Park Press, Baltimore, Md., USA, 1978.

The vaccine is administered at a defined dose in one or more intramuscular or subcutaneous injections, followed by a booster or boosters if required. The immunizing effect of the vaccine is monitored by determination of anti-HBsAgm and/or anti-Pre-Sm protein antibodies in the vaccinated individual. In the case of nucleic acid vaccines, the concentration of nucleic acid in the composition used for administration in vivo is from about 100 μg/ml to 10 mg/ml, preferably 1 mg/ml.

The administration of derived protein(s) or peptide(s) of interest or of their fragment(s), alone or in combination, is used for prophylaxis and/or treatment. These proteins or peptides that are administered are characterized in that they do not exhibit the virulence of HBV but are able to induce a humoral or cellular immune response, in the individual to whom they are administered. Such proteins are called “modified”, but their immunogenicity is conserved. The modified molecules can be obtained by synthetic and/or recombinant techniques or starting from natural molecules modified by chemical or physical treatments.

Vaccinal protein(s) or peptide(s) are identified in the following way: the “modified” candidate molecules are analysed in a functional test to check that they have lost their toxicity and to verify their immunogenicity (i) by conducting an in vitro test of proliferation of CD4+ T lymphocytes specific to the antigen administered (T cell assay) or an in vitro test of cytotoxicity of the CD8+ lymphocytes specific to the antigen administered and (ii) by measuring, among other things, the proportion of circulating antibodies directed against the natural protein. These modified forms are employed for immunizing people by standardized procedures with the appropriate adjuvants.

The nucleic acids for use in vaccines are also analysed (i) by carrying out an in vitro test of proliferation of CD4+ T lymphocytes specific to the antigen administered (T cell assay) or an in vitro test of cytotoxicity of CD8+ lymphocytes specific to the antigen administered and (ii) by measuring, among other things, the proportion of circulating antibodies directed against the protein encoded by the viral DNA.

The vaccines prepared are injectable, i.e. in liquid solution or in suspension. Optionally, the preparation can also be emulsified. The antigenic molecule can be mixed with excipients that are pharmaceutically acceptable and compatible with the active ingredient. Examples of favourable excipients are water, a saline solution, dextrose, glycerol, ethanol or equivalents and their combinations. If desired, the vaccine can contain smaller quantities of auxiliary substances such as wetting agents or emulsifiers, pH buffering agents or adjuvants such as aluminium hydroxide, muramyl dipeptide or variants thereof. In the case of peptides, their coupling to a larger molecule (KLH, tetanic toxin) increases immunogenicity by several times. The vaccines are administered conventionally by injection, for example intramuscular. Other favourable formulations with other routes of administration are suppositories and sometimes oral formulations.

The phrase “pharmaceutically acceptable vehicle” means carriers and vehicles that can be administered to humans or animals, as described for example in Remington's Pharmaceutical Sciences 16th ed., Mack Publishing Co. The pharmaceutically acceptable vehicle is preferably isotonic, hypotonic or exhibits a slight hypertonicity and has a relatively low ionic strength. The definitions of pharmaceutically acceptable excipients and adjuvants are also given in the aforementioned Remington's Pharmaceutical Sciences.

One aspect of the invention also relates to a therapeutic or prophylactic preparation for the treatment or the prevention of infection by the mHBV virus that includes a therapeutic or prophylactic agent, i.e. at least anti-HBsAgm protein and/or anti-Pre-Sm protein antibodies and/or antibodies directed against fragments of the said proteins, optionally combined with anti-wild-type HBsAg and/or anti-wild-type Pre-S protein antibodies and/or antibodies directed against fragments of the said proteins, in particular neutralizing antibodies and their uses for the treatment or the prevention of the disease. Immunoglobulins, whose titre of antibodies, especially of anti-HBs antibodies, hepatitis B controlled, can be used in prophylaxis in subjects not vaccinated against hepatitis B, accidentally contaminated, and in neonates from an infected mother. The definition of therapeutic or prophylactic preparation also includes the aforementioned vaccinal or immunogenic compositions.

The efficacy of a therapeutic or prophylactic agent is evaluated using an animal model. An animal is injected with at least one HBsAgm or Pre-Sm protein of the invention, and preferably both, obtained by isolation and purification from serum or plasma, by genetic recombination or by peptide synthesis, optionally combined with the HBsAgm′ and/or Pre-Sm′ protein and/or with a wild-type HBsAg and/or wild-type Pre-S protein. The injections are made, at various established concentrations, in mammals such as mice or rats, by the intramuscular, subcutaneous or other routes. A negative control is conducted in parallel. The injections are made in a single dose or in repeated doses, with different intervals of time between each administration. A few hours to a few weeks after administration, biological samples are taken, preferably of blood or of serum. The following are performed on these samples:

(i) assay of specific antibodies of the protein(s) or peptide(s) of interest or of their fragments, alone or in combination, and/or

(ii) assay of the cellular immune response induced against the protein(s) or peptide(s) of interest or their fragments and against any immunogenic peptide derived from the said proteins or peptides or their fragments, by conducting, for example, a test of in vitro activation of helper T cells specific to the antigen administered, by quantifying the cytotoxic T lymphocytes in accordance with the so-called ELISPOT technique, described by Scheibenbogen et al., 1997 Clinical Cancer Research 3: 221-226.

The said determination is particularly advantageous for evaluating the efficacy of a vaccinal approach in an individual or for diagnosis and/or prognosis of a potential pathologic state by trying to demonstrate an immune response that would be developed naturally in a patient.

The animal is then sacrificed and the efficacy of the therapeutic agent is demonstrated

(i) by classical immunohistologic analyses using ligands of the proteins of interest and/or of their fragments, in particular monoclonal or polyclonal antibodies or fragments of the said antibodies, and/or

(ii) by classical techniques of in situ hybridization using nucleic acid fragments or oligonucleotides defined on the basis of knowledge of the nucleotide sequences that code for the proteins of interest or for their fragments or on the basis of knowledge of the polypeptide sequences of the said proteins of interest or of their fragments; and/or

(iii) by techniques of in situ PCR amplification using nucleic acid fragments or primers defined on the basis of the nucleotide or polypeptide sequences of the proteins of interest or of their fragments.

Evaluation of the efficacy of a therapeutic or prophylactic agent and therapeutic monitoring ex vivo, in man, is determined in the following way: the therapeutic agents to be tested for therapeutic activity and/or for therapeutic monitoring are administered in man by various routes, such as intramuscular, subcutaneous or other routes. Various doses are administered to human beings. The clinical history of the patient at the time of the first administration is known perfectly. One or more administrations can be effected with different time intervals between each administration ranging from a few days to a few years. Biological samples are taken at defined intervals of time after administration of the therapeutic agent, preferably of blood and of serum. Various analyses are carried out on these samples. Just before the first administration of the therapeutic agent, the said samples are taken and the same analyses are also performed. Classical clinical and biological examination is also carried out in parallel with the supplementary analyses that are described below, at different analysis times. The following analyses are carried out: qualitative and quantitative measurement of the proteins of interest in the serum or in the blood by ELISA and/or Western Blot, using antibodies or antibody fragments that are able to fix to at least one of the proteins or to one of their fragments and/or measurement of the activity of the said proteins and/or assay of antibodies specific to the proteins of interest or of their fragments in the blood or serum samples by ELISA and/or Western Blot using an isolated and purified natural protein or a fragment of the natural protein and/or a recombinant protein or a fragment of the said recombinant protein or a synthetic polypeptide, and/or assay of the cellular immune response induced against the protein or proteins of interest and any immunogenic peptide derived from these proteins, as described previously, and/or detection of DNA and/or RNA fragments coding for the protein or proteins of interest or a fragment of the said proteins of interest by nucleotide hybridization by the techniques that are familiar to a person skilled in the art (Southern blot, Northern blot, ELOSA “Enzyme-Linked Oligosorbent Assay” (Katz J B et al., Am. J. Vet. Res., 1993 Dec; 54 (12): 2021-6 and François Mallet et al., Journal of Clinical Microbiology, June 1993, p. 1444-1449)) and/or by DNA and/or RNA amplification, for example by PCR, RT-PCR, using nucleic acid fragments coding for the protein or proteins of interest, and/or by biopsy of tissues, preferably from the liver, and observation of the characteristic effects of the protein or proteins.

When the therapeutic agent is an antibody, an antibody fragment or a mixture of antibodies and/or antibody fragments, the patient is administered either soluble neutralizing antibodies or antibody fragments for inhibiting protein activity, or specific soluble antibodies or antibody fragments for eliminating the protein by formation of immune complexes. The neutralizing antibodies are polyclonal or monoclonal or are antibody fragments that recognize the active site of the protein and, by attaching themselves, inhibit the function of the protein. The non-neutralizing antibodies are polyclonal or monoclonal antibodies or fragments of the said antibodies that are able to recognize an immunodominant region of the protein and eliminate it by forming an immune complex. The antibody's ability to attach itself specifically to the protein is analysed by conventional techniques that have been described, for example by ELISA or Western Blot tests using the protein or the natural or synthetic immunogenic peptide. The antibody titre is determined. The antibody's ability to neutralize the function of the protein can be analysed in various ways, for example by determining the decrease in activity of the protein or of the immunogenic peptide in the presence of the antibody. Monoclonal or polyclonal antibodies directed against a target protein or a part of the said protein are produced by conventional techniques used for producing antibodies against surface antigens. Mice or rabbits are immunized (i) either with a natural or recombinant protein, (ii) or with any immunogenic peptide derived from the said protein, (iii) or with murine cells that express the protein of interest or the peptide and MHC molecules. The Balb/c murine line is used most often.

The present invention therefore relates to a biological material for preparing a pharmaceutical composition intended for the treatment of humans infected by at least the mHBV virus, the said composition comprising:

(i) either at least one natural protein and/or recombinant protein and/or synthetic polypeptide or their fragments whose sequence corresponds to the whole or part of the sequences with the references SEQ ID NO 2 and 4, independently or in combination, and optionally combined with the whole or part of at least one natural protein and/or recombinant protein and/or polypeptide or their fragments whose sequences have the references SEQ ID NOs 5 and 6 and/or to the whole or part of a natural and/or recombinant protein and/or a synthetic polypeptide or their fragments of wild-type HBV. The present inventors in fact identified, after cloning, sequencing and alignment with the protein sequences available in the NBRF-PIR bank, another variant of genotype D, isolated from the same individual and possessing two significant mutations. The first mutation relates to the amino acid Arg, in position 201 of the HBsAg protein identified in SEQ ID NO 5 and the second mutation relates to the amino acid Gly, in position 102 of the Pre-S region identified in SEQ ID NO 6. The codons coding respectively for these two amino acids are the codons AGG at position 628-630 in the S gene and at position 782-784 relative to the sequence of the complete genome and the codon GGA at position 304-306 in the Pre-S region and at position 3151-3153 relative to the sequence of the complete genome;

(ii) or at least one monoclonal or polyclonal antibody or a fragment of the said antibodies, specific to at least one of the said proteins or its fragments, the said antibodies or fragments being usable alone or in combination and being capable of attaching themselves to at least one of the proteins meeting the above definitions. This antibody can be neutralizing or non-neutralizing, i.e. able or unable to neutralize the protein activity. These antibodies are very useful, notably in that they permit the application of therapeutic compositions since they lead for example to immune reactions, directed specifically against immunodominant epitopes or against the antigens.

The invention also relates to ligands that are capable of binding to a nucleotide sequence of DNA or of RNA or to a nucleotide fragment as defined above. Thus, by ligand we mean any molecule that is able to bind to a nucleotide sequence of DNA or of RNA or to a nucleotide fragment, such as a partially or fully complementary nucleotide fragment, a complementary polynucleotide, an anti-nucleic acid antibody. The production of nucleotide fragments or of polynucleotides is within the general knowledge of a person skilled in the art. We may mention in particular the use of restriction enzymes, and chemical synthesis in an automatic synthesizer. The probes and primers that are capable of hybridization under stringency conditions conditions determined for a nucleotide sequence of DNA or of RNA or for a nucleotide fragment as defined previously are included in this definition. It is within the ability of a person skilled in the art to define the appropriate stringency conditions conditions. Characteristic stringency conditions are those that correspond to a combination of temperature and of saline concentration chosen approximately between 12 to 20° C. below Tₘ (melting temperature) of the hybrid under investigation. The stringency conditions for discriminating even a single point mutation have been known since at least the year 1979. The following may be cited as examples: Wallace R. B. et al., DNA. Nucleic Acids Res., 6, 3543-3557 (1979), Wallace R. B. et al., Science, 209, 1396-1400 (1980), Itakura K. and Riggs A. D., Science, 209, 1401-1405 (1980), Suggs S. V. et al., PNAS, 78, 6613-6617 (1981), Wallace R. B. et al., DNA, Nucleic Acids Res., 9, 3647-3656 (1981), Wallace R. B. et al., DNA, Nucleic Acids Res., 9, 879-894 (1981) and Conner B. J. et al., PNAS, 80, 278-282 (1983). Furthermore, techniques are known for the production of anti-nucleic acid antibodies. As examples we may cite Philippe Cros et al., Nucleic Acids Research, 1994, Vol. 22, No. 15, 2951-2957; Anderson, W. F. et al. (1988) Bioessays, 8 (2), 69-74; Lee, J. S. et al. (1984) FEBS Lett., 168, 303-306; Malfoy, B. et al. (1982) Biochemistry, 21 (22), 5463-5467; Stollar, B. D. et al., J. J. (eds) Methods in Enzymology, Academic Press, pp. 70-85; Traincard, F. et al. (1989) J. Immunol. Meth., 123, 83-91 and Traincard, F. et al. (1989) Mol. Cell. Probes, 3, 27-38).

By nucleotide fragment we mean either fragments bound to a same molecular unit, or fragments in a molecular complex comprising several homologous or heterologous subunits obtained naturally or artificially, especially by multiple differential splicing or by selective synthesis.

We thus define a diagnostic composition that includes at least one probe or one primer or one anti-nucleic acid antibody.

In addition, the primers, probes and anti-nucleic acid antibodies of the invention are used in a method for diagnosis of viral DNA and/or RNA, according to which a sample of serum or plasma is taken from a patient, the said sample is treated if necessary to extract the DNA and/or the RNA, the said sample is brought into contact with at least one probe or one primer or one anti-nucleic acid antibody as defined previously, under stringency conditions conditions determined when the ligand is a probe or a primer, and the presence of viral DNA and/or RNA in the sample is detected either by demonstrating hybridization of the said viral DNA and/or RNA with at least one probe, or by amplification of the said DNA and/or RNA, or in conditions of incubation determined when the ligand is an anti-nucleic acid antibody and the complex thus formed is detected. When using an anti-nucleic acid antibody, the antibody itself can be labelled with any suitable marker for detecting the complex formed, or also the formation of the complex can be detected by adding an antibody to the anti-labelled nucleic acid-antibody to the incubation medium. When probes are used, the presence of the hybridization complex can be demonstrated directly by using a probe that is complementary or approximately complementary to the sequence of the target, the said probe being labelled with any suitable marker or by applying the so-called “sandwich” technique in one or two stages, which consists of using a capturing probe that is complementary or approximately complementary to a portion of the sequence of the target and a labelled “detection” probe that is complementary or approximately complementary to another portion of the target sequence. In the case when primers are used, these can be labelled directly for detecting an amplification product.

The present invention also relates to a biological material for the preparation of pharmaceutical compositions intended for the treatment of an infection at least by mHBV, the said composition comprising (i) either at least one nucleic acid sequence able to hybridize with at least one of the nucleic acid sequences SEQ ID NO 1 and SEQ ID NO 3 or their complementary sequences, especially with the nucleic acid sequence SEQ ID NO 1 or its complementary sequence, optionally in combination with a nucleic acid sequence that can hybridize with at least one of the nucleotide sequences coding for SEQ ID NO 5 and for SEQ ID NO 6 or their complementary sequences and/or with at least one nucleic acid sequence of wild-type HBV; or fragments of the aforementioned sequences, (ii) or at least one nucleic acid sequence containing at least one gene of therapeutic interest and elements ensuring expression of the said gene in vivo in target cells intended to be genetically modified by the said nucleic acid sequence, (iii) or at least one mammalian cell not naturally producing the protein or proteins of the invention or their fragments or specific antibodies of at least one of the said proteins or its fragments; the said mammalian cell being genetically modified in vitro by at least one nucleic acid sequence or a fragment of a nucleic acid sequence or a combination of nucleic acid sequences corresponding to nucleic acid fragments obtained from a same gene or from different genes, the said gene of therapeutic interest coding for all or part of the protein or proteins of interest or their fragment(s) or for a specific antibody of the protein or proteins of interest that is to be expressed on the surface of the said mammalian cell (Toes et al., 1997, PNAS 94: 14660-14665). The pharmaceutical composition can contain a single therapeutic agent directed against a single target or agents used in combination directed against several targets.

Thus, the present invention also relates to a biological material for the preparation of pharmaceutical compositions comprising at least one nucleic acid sequence able to hybridize with a nucleic acid sequence as defined above.

The nucleic acid sequences and/or vectors (antisense or coding for a protein) make it possible in particular to target the cells in which a gene is expressed.

Nucleic acid sequences or antisense oligonucleotides are able to interfere specifically with the synthesis of a target protein of interest, by inhibiting the formation and/or the functioning of the polysome, depending on the location of the antisense in the target's mRNA. Therefore the frequent choice of the sequence surrounding the translation initiating codon as target for inhibition by an antisense oligonucleotide aims to prevent the formation of the initiation complex. Other mechanisms in inhibition by antisense oligonucleotides involve activation of ribonuclease H that digests the antisense oligonucleotide/mRNA hybrids or interference at splicing sites by antisense oligonucleotides whose target is an mRNA splicing site. The antisense oligonucleotides are also complementary to DNA sequences and so can interfere at the transcription level by forming a triple helix, the antisense oligonucleotide pairing by so-called Hoogsteen hydrogen bonds in the major groove of the DNA double helix. In this special case, it is more accurate to call them antigenic oligonucleotides. The antisense oligonucleotides can of course be strictly complementary to the DNA or RNA target with which they must hybridize, but also not strictly complementary on the condition that they hybridize with the target. Moreover, they may be antisense oligonucleotides that are unmodified, or are modified at the level of the inter-nucleotide bonds. All these concepts are included in the general knowledge of a person skilled in the art.

The present invention therefore relates to a pharmaceutical composition comprising, inter alia, a nucleic sequence or antisense oligonucleotide as defined above.

The present invention also relates to the use of vectors comprising at least one gene of therapeutic interest in relation to the genes of the proteins of interest identified in the present invention and a biological material for the preparation of pharmaceutical compositions intended for treating patients infected with at least the mHBV virus, the said composition comprising a nucleic acid sequence including a gene of therapeutic interest and elements for expressing the said gene of interest. The genes can be unmutated or mutated. They can also consist of nucleic acids modified so that they are unable to integrate in the genome of the target cell or nucleic acids stabilized by means of agents, such as spermine.

Such a gene of therapeutic interest codes in particular:

(i) either for at least one protein or protein fragment of the invention;

(ii) or for at least all or part of a polyclonal or monoclonal antibody that is able to attach itself to at least one protein of the present invention. In particular this may be a native transmembrane antibody, or a fragment or derivative of such an antibody, provided the said antibody, or antibody fragment or derivative is expressed on the surface of a target cell of a mammal genetically modified for the purposes of the present invention and is able to attach itself to a polypeptide present on the surface of a cytotoxic effector cell or a helper T lymphocyte involved in the process of activation of such a cell;

(iii) or for at least one inhibitor molecule of at least one protein of the invention;

(iv) or for at least one ligand or any part of a ligand that is able to attach itself to at least one protein or protein fragment of the invention and/or inhibit its function.

By transmembrane antibody we mean an antibody of which at least the functional region capable of recognizing and attaching itself to its specific antigen is expressed on the surface of the target cells to permit the said recognition and attachment. More particularly, the antibodies according to the present invention consist of fusion polypeptides containing the amino acids defining the said functional region and an amino acid sequence (transmembrane polypeptide) permitting anchoring within the membrane lipid bilayer of the target cell or to the exterior surface of this bilayer. The nucleic sequences coding for numerous transmembrane polypeptides are described in literature.

“Elements ensuring expression of the said gene in vivo” refers in particular to the elements necessary for ensuring expression of the said gene after its transfer into a target cell. It applies in particular to promoter sequences and/or regulating sequences that are effective in the said cell, and optionally the sequences required to permit a polypeptide to be expressed on the surface of the target cells. The promoter used can be a viral promoter, ubiquitous or tissue-specific, or a synthetic promoter. As examples we may mention the promoters, such as the promoters of the viruses RSV (Rous Sarcoma Virus), MPSV, SV40 (Simian Virus), CMV (Cytomegalovirus) or of the vaccinia virus. In addition it is possible to select a promoter sequence specific to a given cell type, or that can be activated in defined conditions. Literature contains a large volume of information concerning the said promoter sequences.

According to one embodiment of the invention, the therapeutic gene consists of a nucleic acid sequence of naked DNA or RNA, i.e. free from any compound facilitating its introduction into cells (nucleic acid sequence transfer). However, in order to promote its introduction into the target cells and obtain the genetically modified cells of the invention, the nucleic acid sequence can be in the form of a “vector”, and more especially in the form of a viral vector, for example an adenoviral or retroviral vector, a vector derived from a poxvirus, in particular derived from the vaccinia virus or from the Modified Virus Ankara (MVA) or from a non-viral vector, for example a vector consisting of at least one nucleic acid sequence complexed or conjugated with at least one carrier molecule or substance. Literature contains a large number of examples of these viral and non-viral vectors.

Such vectors can moreover and preferably include targeting elements that can permit the transfer of nucleic acid sequences to be directed towards certain cell types or certain particular tissues, such as cytotoxic cells and antigen-presenting cells. They can also permit the transfer of an active substance to be directed towards certain preferred intracellular compartments, such as the nucleus or the peroxisomes. It may also be a question of elements facilitating penetration to the interior of the cell or lysis of intracellular compartments. These targeting elements are widely described in literature. It may be a question, for example, of the whole or part of peptides, oligonucleotides, antigens, antibodies, specific ligands of membrane receptors, and ligands capable of reacting with an anti-ligand, alone or in combination.

The present invention relates to a biological material for the preparation of pharmaceutical compositions comprising at least one vector containing a therapeutic gene, capable of being introduced into a target cell in vivo and of expressing the gene of therapeutic interest in vivo. The advantage of this invention resides in the possibility of maintaining, over the long term, a base level of molecules expressed in the treated patient. Vectors or nucleic acids coding for genes of therapeutic interest are injected. These vectors and nucleic acids must be transported to the target cells and must transfect these cells, in which they must be expressed in vivo.

The invention also relates to the expression in vivo of nucleotide sequences and/or of vectors as described in the preceding paragraph, i.e. sequences corresponding to genes of therapeutic interest coding in particular for:

(i) either at least one protein of the invention, or its fragments,

(ii) or at least all or part of a polyclonal or monoclonal antibody capable of attaching itself to at least one protein of the invention. It may be a native transmembrane antibody, or a fragment or derivative of such an antibody, provided that the said antibody, or antibody fragment or derivative is expressed on the surface of the target cell of a genetically modified mammal and in that the said antibody is capable of attaching itself to a polypeptide present on the surface of a cytotoxic effector cell or a helper T lymphocyte and involved in the process of activation of such a cell. It may be a question of antibody fragments expressed by cells capable of secreting the said antibodies in the blood circulation of a mammal that is a carrier of cells genetically modified by the gene coding for the antibody, either at least for an inhibitory molecule of at least one protein chosen from the proteins of the invention, or at least for a ligand or any part of the ligand capable of attaching to at least one protein of the invention, and/or of inhibiting its function.

According to a particular embodiment, gene therapy is employed so as to direct the immune response against at least one protein, especially HBsAgm, of the invention, and/or against any molecule that inhibits the function and/or expression and/or metabolism of at least one protein of the invention, and/or against ligands of at least one of the proteins of the invention, in particular against one or more receptors. For this, it is obvious that the cells to be targeted for transformation with a vector are cells belonging to the immune system, or cells of the lymphocyte type (CD4/CD8), or antigen-presenting cells.

According to a particular embodiment, the antigen-presenting cells (APCs) are modified genetically, especially in vivo. The APCs, such as macrophages, dendritic cells, microgliocytes, and astrocytes, play a role in the initiation of the immune response. They are the first cell components to capture the antigen, prime it intracellularly and express class I MHC and class II MHC transmembrane molecules involved in presenting the immunogen to the CD4+ and CD8+ T cells, they produce specific auxiliary proteins that take part in activation of the T cells (Debrick et al., 1991, J. Immunol. 147: 2846; Reis et al., 1993, J Ep Med 178: 509; Kovacsovics-bankowski et al., 1993, PNAS 90: 4942; Kovacsovics-bankowski et al., 1995 Science 267: 243; Svensson et al., 1997, J Immunol 158: 4229; Norbury et al., 1997, Eur J Immunol 27: 280). For vaccination, it may be advantageous to have at our disposal a system of gene therapy that can target gene transfer into the said antigen-presenting cells, i.e. a gene that codes for a polypeptide which can, after its intracellular production and its transformation, be presented to the CD8+ and/or CD4+ cells by the class I MHC and class II MHC molecules respectively on the surface of these cells.

We choose to express, on the surface of the antigen-presenting cells in vivo, all or part of an antibody and/or of a ligand such as a receptor for example, capable of reacting with a protein of the invention. The said cells will then specifically phagocytize the protein, and transform it in such a way that fragments are presented to the surface of the antigen-presenting cells.

A great many examples of genes coding for antibodies capable of reacting with polypeptides or receptors are proposed in literature. A person skilled in the art would be able to obtain the nucleic acid sequences coding for the said antibodies. We may mention for example the genes coding for the light and heavy chains of the antibody YTH 12.5 (anti-CD3) (Routledge et al., 1991, Eur J Immunol 21: 2717-2725), of the anti-CD3 according to Arakawa et al., 1996, J. Biochem. 120: 657-662. The nucleic acid sequences of these antibodies are readily identifiable from the databases commonly employed by a person skilled in the art. It is also possible, starting from hybridomas available from the ATCC, to clone the nucleic acid sequences coding for the heavy and/or light chains of these various antibodies by amplification techniques such as RT-PCR using specific oligonucleotides or techniques employing cDNA banks (Maniatis et al., 1982, Molecular cloning. A laboratory manual. CSH Laboratory, Cold Spring Harbor, N.Y.). The sequences thus cloned are then available to be cloned in vectors. According to a preferred case of the invention, the nucleic acid sequence coding for the heavy chain of the antibody is fused by homologous recombination with the nucleic acid sequence coding for a transmembrane polypeptide such as rabic glycoprotein or gp160 (Polydefkis et al., 1990, J Exp Med 171: 875-887). These techniques of molecular biology have been described perfectly well.

We choose to express, on the surface of the antigen-presenting cells in vivo, immunogenic fragments corresponding to at least one protein of the invention. For this, we can choose to use the vector to express either a complete polypeptide, or polypeptides selected for reacting with ligands and/or specific receptors. The immunogenic peptide encoded by the nucleic acid or the polynucleotide introduced into the cell of the vertebrate in vivo can be produced and/or secreted, prepared then presented to an antigen-presenting cell (APC) in the context of the MHC molecules. The APCs thus transferred in vivo induce an immune response directed against the immunogen expressed in vivo. The APCs possess various mechanisms for capturing the antigens: (a) capture of antigens by membrane receptors such as immunoglobulin receptors (Fc) or for complement, available on the surface of the granulocytes, monocytes or macrophages permitting efficient delivery of the antigen to the intracellular compartments after receptor-mediated phagocytosis. (b) entry into the APCs by fluid-phase pinocytosis.

According to a particular embodiment, the cytotoxic effector cells or the helper T lymphocytes are modified genetically, especially in vivo, so that they express, on their surface, a polypeptide or one or more ligands of the said polypeptide, not expressed naturally by these cells, and able to induce their activation, by introducing, into these cells, nucleic acid sequences containing the gene coding for the polypeptide.

In accordance with the present invention, it is also possible to select a nucleic acid sequence containing a gene of therapeutic interest coding for all or part of an antibody directed against at least one protein of the invention and capable of being expressed on the surface of the target cells of the patient to be treated, the said antibody being capable of attaching itself to a polypeptide that is not expressed naturally by the cytotoxic effector cells or helper T lymphocytes.

“Cytotoxic effector cells” means macrophages, astrocytes, cytotoxic T lymphocytes (TCLs) and killer cells (NKs) as well as their derivatives, for example the LAK (Versteeg 1992 Immunology today 13: 244-247; Brittende et al. 1996, Cancer 77: 1226-1243). “Helper T lymphocytes” means in particular the CD4 which permit, after activation, the secretion of factors activating the effector cells of the immune response. The polypeptides and especially the receptors that are expressed on the surface of these cells and are involved in the activation of the said cells consist in particular, wholly or partly of the TCR complex or CD3, wholly or partly, of the complexes CD8, CD4, CD28, LFA-1, 4-1BB (Melero et al., 1998, Eur J Immunol 28: 1116-1121), CD47, CD2, CD1, CD9, CD45, CD30, CD40, wholly or partly of the cytokine receptors (Finke et al., 1998, Gene therapy 5: 31-39), such as IL-7, IL-4, IL-2, IL-15 or GM-CSF, wholly or partly of the receptor complex of the NK cells, for example NKAR, Nkp46, etc. (Kawano et al., 1998 Immunology 95: 5690-5693; Pessino et al., 1998, J Exp Med 188: 953-960), Nkp44, all or part of the receptors of macrophages, for example the Fc receptor (Deo et al., 1997, Immunology Today 18: 127-135).

Numerous tools have been developed for introducing various heterologous genes and/or vectors into cells, especially mammalian cells. These techniques can be divided into two categories: the first category involves physical techniques such as micro-injection, electroporation or particle bombardment. The second category is based on the use of techniques in molecular and cell biology by which the gene is transferred with a biological or synthetic vector that facilitates the introduction of the material into the cell in vivo. At present the most efficient vectors are the viral vectors, especially the adenoviral and retroviral vectors. These viruses possess natural properties for crossing plasma membranes, avoiding degradation of their genetic material and introducing their genome into the cell nucleus. These viruses have been studied extensively and some are already being used experimentally in human applications in vaccination, in immunotherapy, or for compensating genetic deficiencies. However, this viral approach has limitations, due in particular to restricted capacity for cloning in these viral genomes, the risk of spreading the viral particles produced in the organism and the environment, the risk of artefact mutagenesis by insertion in the host cell in the case of retroviruses, and the possibility of inducing a strong inflammatory immune response in vivo during treatment, which limits the possible number of injections (McCoy et al., 1995, Human Gene Therapy 6: 1553-1560; Yang et al., 1996, Immunity 1: 433-422). Alternatives to these viral vector systems exist. The use of non-viral methods, for example co-precipitation with calcium phosphate, the use of receptors that mimic the viral systems (for a summary see Cotten and Wagner 1993, Current Opinion in Biotechnology, 4: 705-710), or the use of polymers such as polyamidoamines (Haensler and Szoka 1993, Bioconjugate Chem., 4: 372-379). Other non-viral techniques are based on the use of liposomes, whose efficacy for the introduction of biological macromolecules such as DNA, RNA, proteins or pharmaceutically active substances has been widely described in scientific literature. In this area, teams have proposed the use of cationic lipids having a strong affinity for the cell membranes and/or nucleic acids. In fact it has been shown that a nucleic acid molecule itself was able to cross the plasma membrane of certain cells in vivo (WO 90/11092), the efficacy depending in particular on the polyanionic nature of the nucleic

acid. Since 1989 (Felgner et al., Nature 337: 387-388) cationic lipids have been proposed for facilitating the introduction of large anionic molecules, which neutralizes the negative charges of these molecules and favours their introduction into the cells. Various teams have developed cationic lipids of this kind: DOTMA (Felgner et al., 1987, PNAS 84: 7413-7417), DOGS or Transfectam™ (Behr et al., 1989, PNAS 86: 6982-6986), DMRIE and DORIE (Felgner et al., 1993 methods 5: 67-75), DC-CHOL (Gao and Huang 1991, BBRC 179: 280-285), DOTAP™ (McLachlan et al., 1995, Gene therapy 2: 674-622) or Lipofectamine T, and the other molecules described in patents WO9116024, WO9514651, WO9405624. Other groups have developed cationic polymers which facilitate the transfer of macromolecules especially anionic macromolecules into cells. Patent WO95/24221 describes the use of dendritic polymers, document WO96/02655 describes the use of polyethyleneimine or polypropyleneimine and documents U.S. Pat. No. 5,595,897 and FR2719316 describe the use of polylysine conjugates.

It being given that we wish to obtain in vivo a transformation targeted to a given cell type, it is obvious that the vector used must itself be able to be “targeted”.

The present invention also relates to a biological material for the preparation of pharmaceutical compositions, the composition comprising at least one cell, in particular a cell that does not produce antibodies naturally, in a form permitting its administration in a mammalian, human or animal, organism, as well as its prior culture if necessary, the said cell being genetically modified in vitro by at least one nucleic acid sequence containing at least one therapeutic gene coding in vivo for at least one protein or a fragment of a protein of the invention or for at least one molecule that inhibits the function and/or the fixation and/or the expression of at least one protein or one protein fragment of the invention or for at least one antibody or part of an antibody capable of binding to at least one protein of the invention.

More especially, the said target cell originates either from the individual to be treated, or from another mammal. In the latter case, it should be noted that the said target cell will have been treated to make it compatible with humans. These cells are established in cell lines and are preferentially MHC II+ or MHC II+ inducible, such as lymphocytes, monocytes, astrocytes, oligodendrocytes, etc.

The invention also relates to modified cells and a method of preparation of a cell as described above, characterized in that at least one nucleic acid sequence containing at least one gene of therapeutic interest and elements ensuring expression of the said gene in the cell are introduced into a mammalian cell that does not produce antibodies naturally, by any appropriate means, the said gene of therapeutic interest containing a nucleic acid sequence coding for a molecule or a fragment of a molecule in vivo, as described above. More especially, it relates to eukaryotic cells, especially COS and CHO cells and cells obtained from lower eukaryotic organisms, such as yeast cells, especially cells obtained from Saccharomyces cerevisiae and from Pichia pastoris, especially cells transformed by at least one nucleotide sequence and/or a vector as described previously.

According to a particular embodiment, the cells (dendritic cells, macrophages, astrocytes, CD4+ T lymphocytes, CD8+ T lymphocytes or others) from the patient or allogenic cells are placed in contact with a purified preparation of at least one protein or protein fragment of the invention. The protein or its fragment is internalized, prepared and presented on the surface of the cell and associated with MHC I and/or MHC II molecules for inducing a specific immune response against the protein or its fragment. The cells thus “activated” are then administered to the patient in whom they will induce an antigen-specific immune response.

In a special case, the antigen-presenting cells are modified in vitro to express the antigens in the transformed cell that will be associated with the MHC I and/or MHC II molecules and will be presented on the surface of the cells to induce a perfectly targeted immune reaction in the patient to whom the modified cell is administered.

Vaccinal approaches are not always entirely satisfactory and can lead to limited immune reactions directed solely against immunodominant epitopes. Moreover, incorrect presentation of the antigens by the glycoproteins of the MHC system on the surface of the cells does not permit an appropriate anti-protein immunity to develop in the patient treated. In order to alleviate these problems, authors have proposed, within the framework of vaccinal methods, selection of minimal antigenic fragments corresponding to the portions of the peptide that are able to be recognized specifically by the cytotoxic T lymphocytes, and their expression in the cells so that they associate with the MHC I molecules and are presented on the surface of the cells in order to induce a perfectly targeted immune reaction in the treated patient (Toes et al., 1997, PNAS 94: 14660-14665). More especially, it has been shown that very small epitopes (ranging from 7 to about 13 amino acids) that are expressed from minigenes introduced in a vaccinia virus, could induce immunization of the cellular type. It has been shown, moreover, that several minigenes could be expressed together from the same vector (this special construction is called “string of beads”). Such a construction offers the advantage of inducing an immune reaction of synergic CTL type (Whitton et al., 1993, J. of Virology 67: 348-352).

Presentation of the antigenic fragments by the MHC I molecules is based on an identified intracellular method (see Grottrup et al., 1996 Immunology Today 17: 429-435 for a review) in the course of which very short antigenic peptides (about 7 to 13 or 8 to 12 amino acids) are produced by degradation of a more complex polypeptide against which the final immune reaction will be directed. These short peptides are then associated with the MHC I or MHC II molecules to form a protein complex which is transported to the cell surface in order to present the said peptides to the circulating cytotoxic T lymphocytes or to the circulating helper T lymphocytes, respectively.

According to a particular embodiment, the cells, such as dendritic cells, macrophages, astrocytes, CD4+ T lymphocytes, CD8+ T lymphocytes, are modified so as to express specific antibodies of the targeted peptide on their surface. The peptide is neutralized by the antibodies expressed on the surface of the cells. These cells, which preferably were taken from the patient, are cells of the immune system, preferably cytotoxic, modified for expressing the whole or part of a specific antibody of the target polypeptide.

In 1968, Boyum described a rapid technique that makes it possible, by density-gradient centrifugation of the blood, to separate the mononuclear cells (lymphocytes and monocytes) at a good yield (theoretical yield 50%, i.e. 10⁶ cells/ml of blood). According to this protocol, 50 ml of peripheral blood obtained in sterile conditions is centrifuged for 20 minutes at 150 g at 20° C. The recovered cells are diluted in two volumes of initial peripheral blood of sterile PBS. 10 ml of this suspension is deposited on 3 ml of a Ficoll-Hypaque solution (lymphocyte separating medium, Flow). After centrifugation for 20 minutes at 400 g and 20° C. without braking for deceleration, the mononuclear cells sediment at the PBS-Ficoll interface, in a dense, opalescent layer, whereas nearly all of the red cells and polynuclears form a sediment at the bottom of the tube. The mononuclear cells are recovered and washed in sterile PBS.

The antigen-presenting cells are first washed with a PBS-BSA buffer at 0.5% (w/v), then counted. Then they are pre-incubated in the presence of various reduction inhibitors, three times in PBS-BSA 0.5% containing from 10 μM to 10 mM (final) of DTNB (5,5′-dithio-bis-2-nitrobenzoic acid) or of NEM (N-ethylmaleimide). The next stages of fixation of antigens on the cell surface or internalization of antigens are also effected in the presence of various concentrations of inhibitors.

8.10⁶ cells are internalized in the presence of a saturating quantity of proteins radiolabelled with iodine-125 (1 μg) in microwells. After incubation for one hour at 4° C. with stirring, the antigens are fixed on the surface of the cells. The cellular suspension is washed twice in PBS-BSA and the cellular residues are taken up in 70 μl of buffer and incubated at 37° C. for various periods ranging up to 2 hours. The cells and supernatants are separated by centrifugation at 800 g for 5 minutes at 4° C. For longer incubation times, the preliminary stage of pre-fixation of the antigens on the surface of the cells is omitted. The cells are diluted in RPMI-10% SVF medium in the presence of 20 mM Hepes, to 10⁶ cells/ml. The cells are incubated in the presence of an excess of antigen for various lengths of time at 37° C. (1 μg of molecules/5.10⁷ monocyte/macrophage cells or/10⁸ B-EBV cells).

All of the therapeutic agents defined in the scope of the present invention are used for preventing and/or treating an infection with at least the mHBV virus. They can also be used for evaluating their efficacy in vitro or in vivo.

The biological material is administered in vivo especially in injectable form by the intramuscular or subcutaneous route or any other equivalent means. Administration can take place in a single or repeated dose, once or several times after a certain interval of time. The best appropriate route of administration and dosage vary depending on a number of parameters such as the individual, the stage and/or development of the disease, or depending on the nucleic acid and/or the protein and/or peptide and/or molecule and/or cell to be transferred or the target organ/tissues.

For carrying out the treatment, it is possible to use pharmaceutical compositions containing a biological material as described previously, advantageously associated with a pharmaceutically acceptable vehicle for administration to humans or animals. The use of these vehicles is described in literature (see for example Remington's Pharmaceutical Sciences 16th ed. 1980, Mack Publishing Co.). The said pharmaceutically acceptable vehicle is preferably isotonic, hypotonic or exhibits a slight hypertonicity and has a relatively low ionic strength, for example a sucrose solution. Furthermore, the said composition can contain solvents, aqueous or partially aqueous vehicles such as sterile water, free from pyrogenic agents and dispersion media for example. The pH of these pharmaceutical compositions is suitably adjusted and buffered in accordance with conventional techniques.

The invention therefore also relates to (i) a method of treating a patient infected with the mHBV virus of the invention in accordance with which the said patient is administered a biological material as defined previously, if necessary combined with an adjuvant and/or a diluent and/or an excipient and/or a pharmaceutically acceptable vehicle and (ii) a method of preventing infection with at least the mHBV virus of the invention in accordance with which an individual is administered a biological material as defined above, if necessary combined with an adjuvant and/or a diluent and/or a pharmaceutically acceptable vehicle, especially a vaccinal composition.

Finally, the invention relates to a composition comprising a DNA sequence coding for a mutated surface protein of the mHBV virus (HBsAgm) or its fragments, the said protein HBsAgm containing a modified determinant a of a protein HBs shown in SEQ ID NO 2, the said DNA being mixed with a vehicle and/or an adjuvant and/or an excipient and/or a suitable diluent and it may also contain a mutated coding sequence Pre-S that codes for a mutated surface protein, referenced in SEQ ID NO 4 and/or a DNA sequence coding for a mutated surface protein referenced in SEQ ID NO 5 or its fragments and/or a DNA sequence coding for a mutated region Pre-S referenced in SEQ ID NO 6 or its fragments.

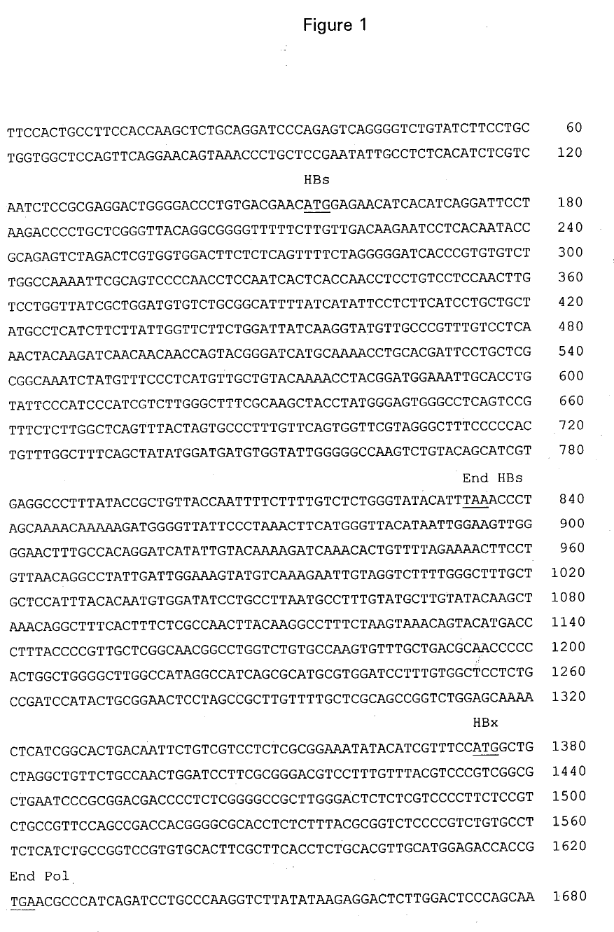

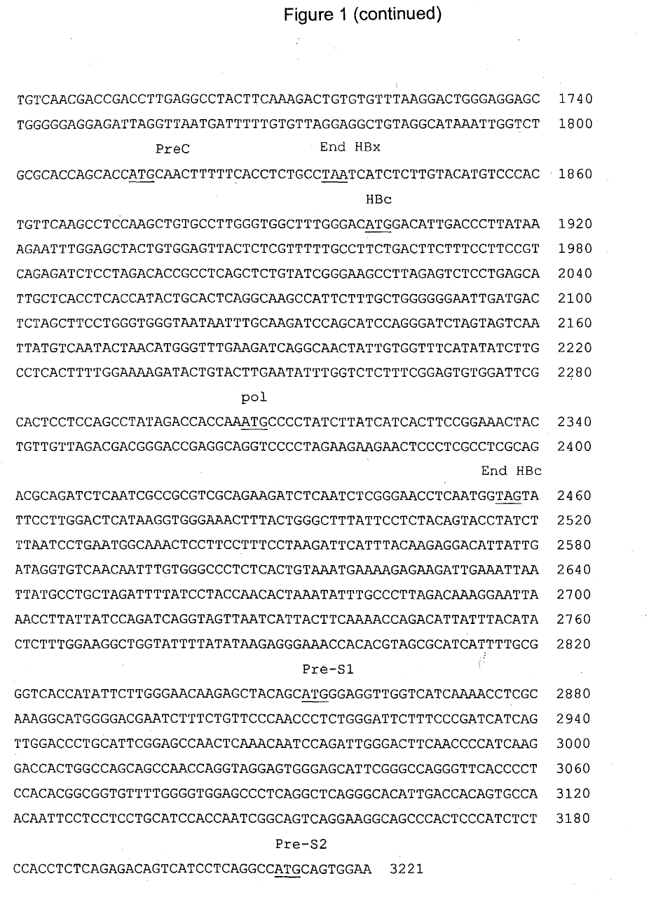

FIG. 1 shows the complete sequence of the clone x27_{—}16 of genotype A of the invention. The underlined sequences correspond to the initiation and termination codons for the various genes.

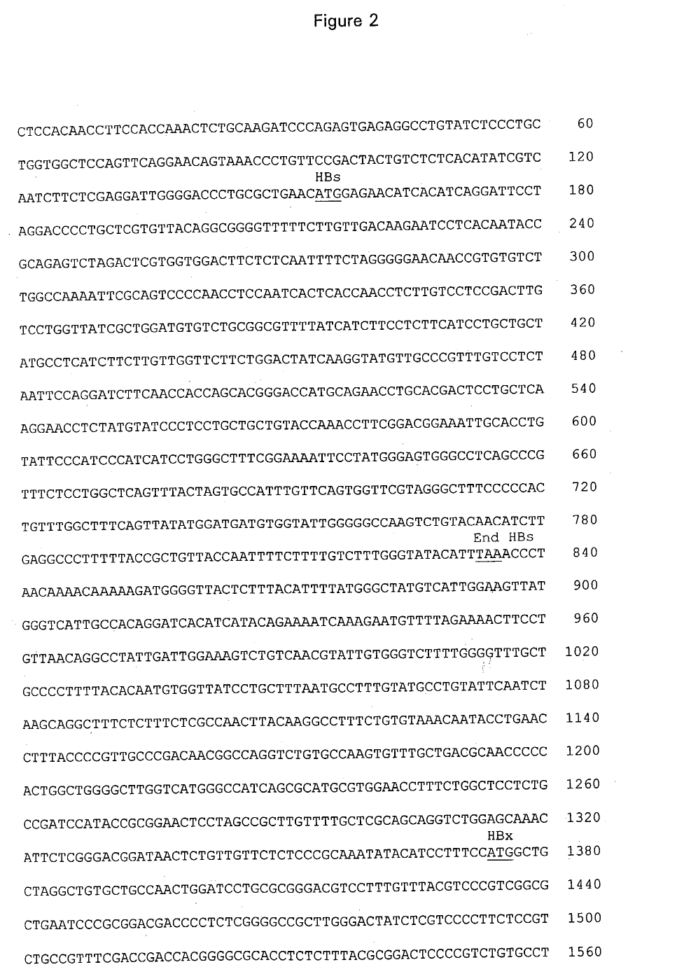

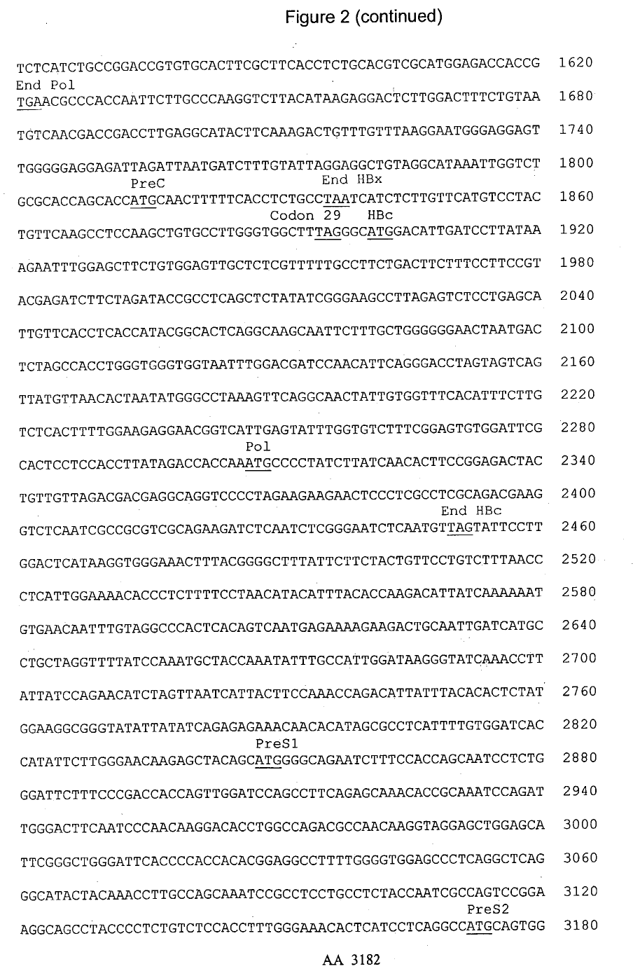

FIG. 2 shows the complete sequence of the clone x27_{—}9 of genotype D of the invention. The underlined sequences correspond to the initiation and termination codons for the various genes.

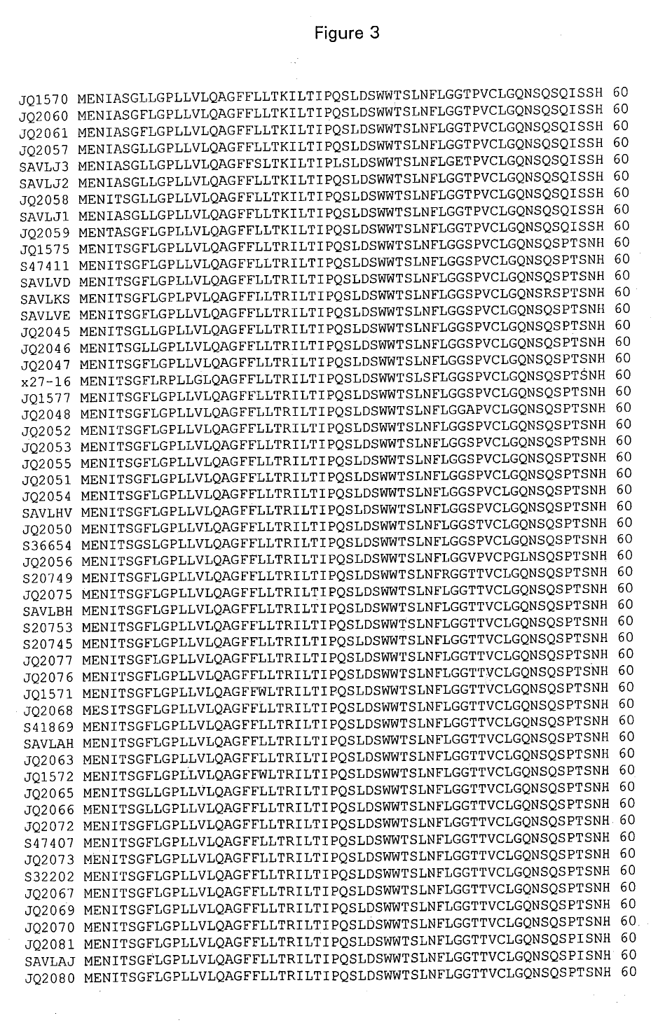

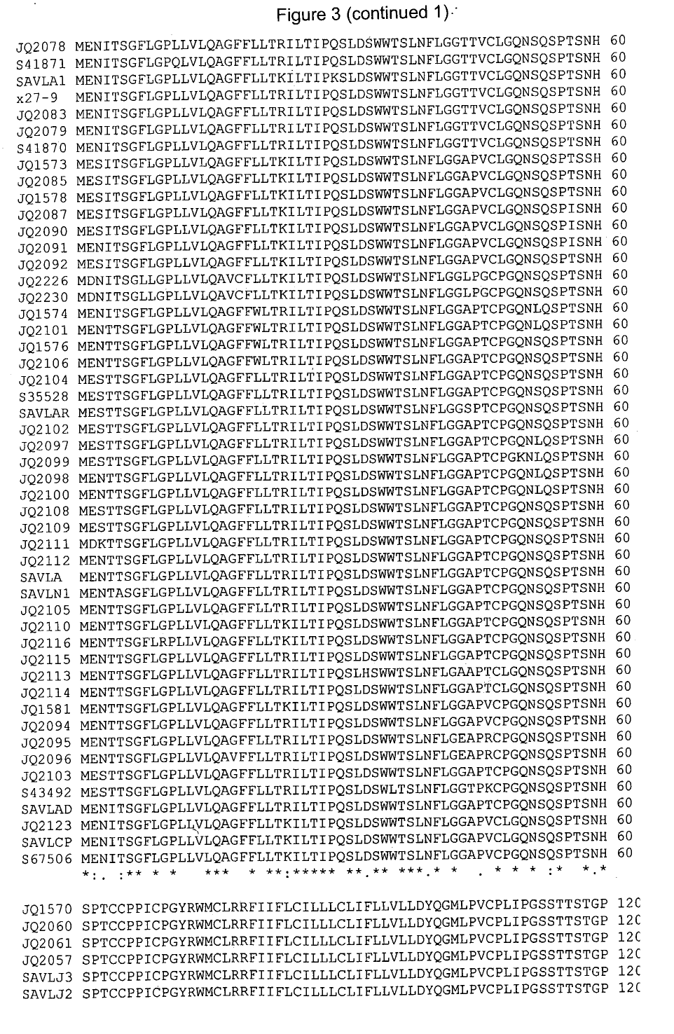

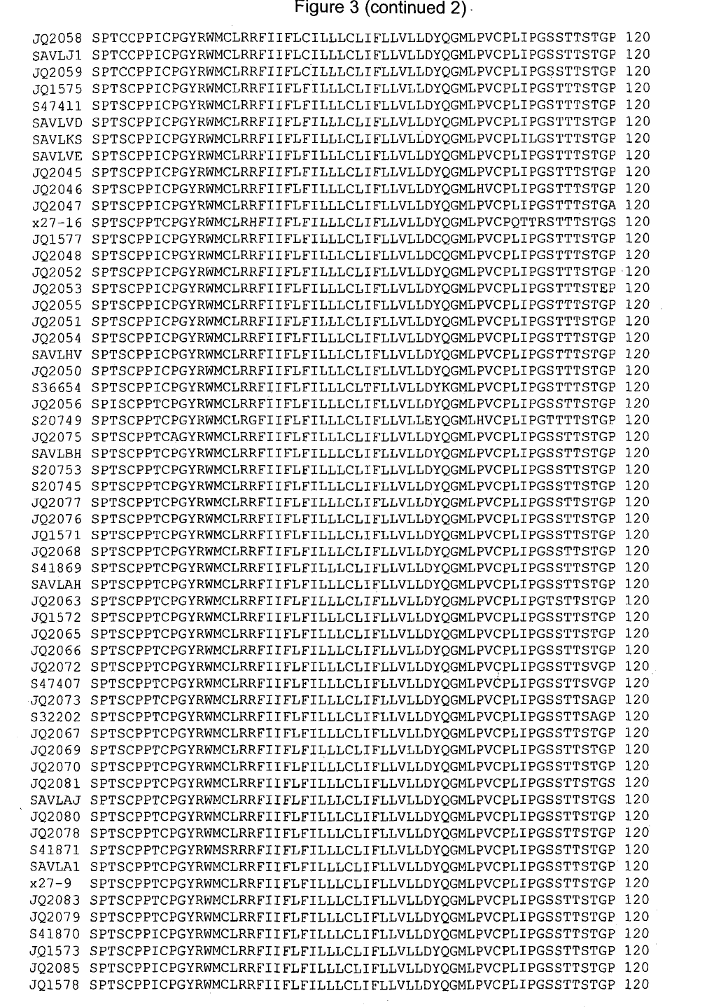

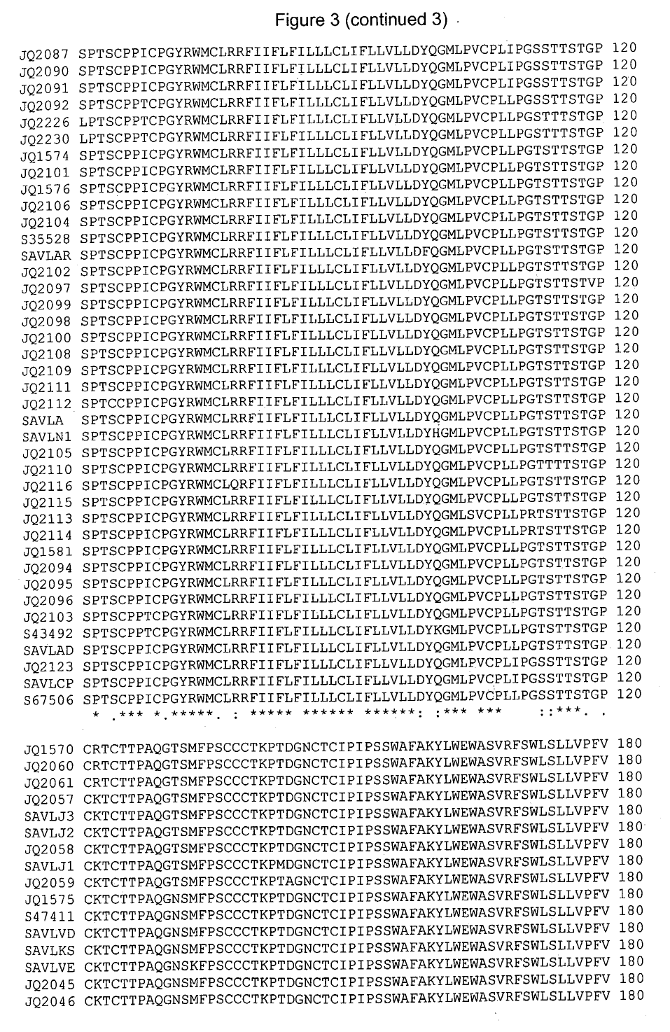

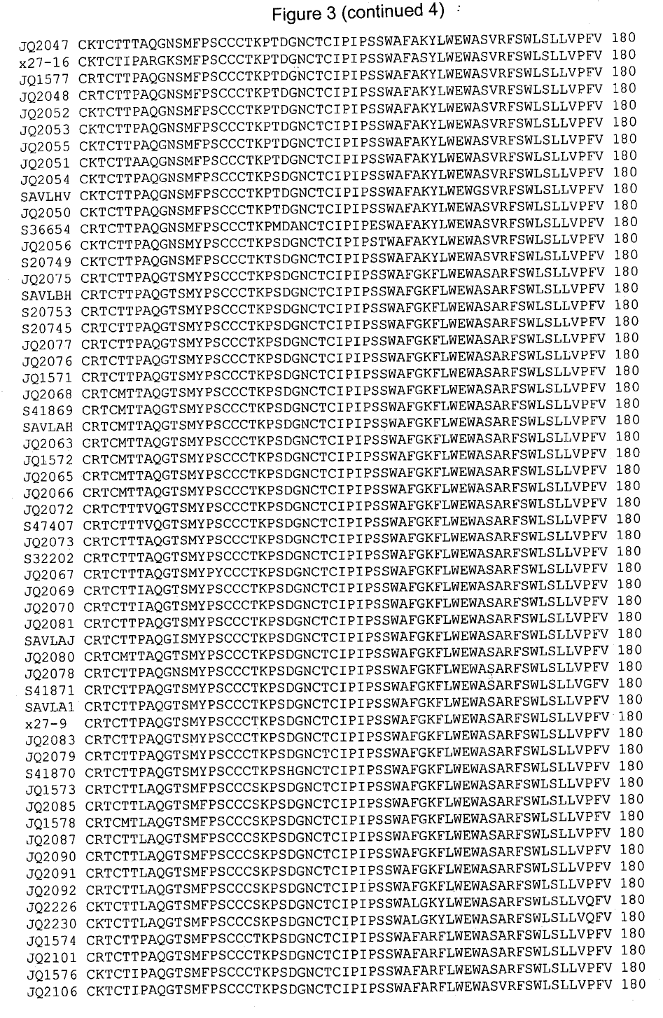

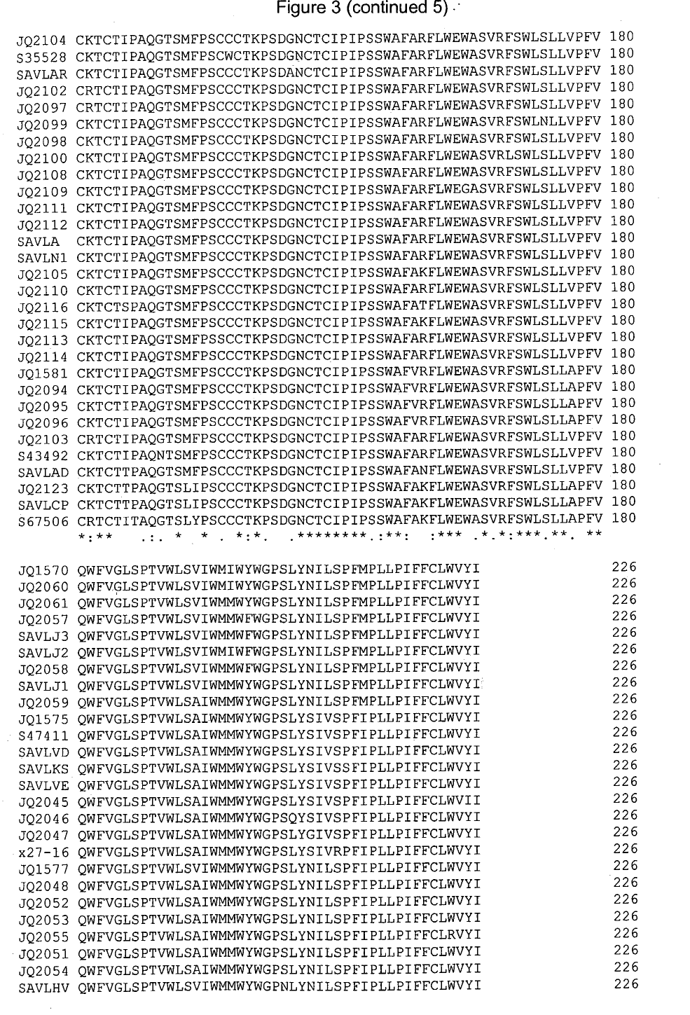

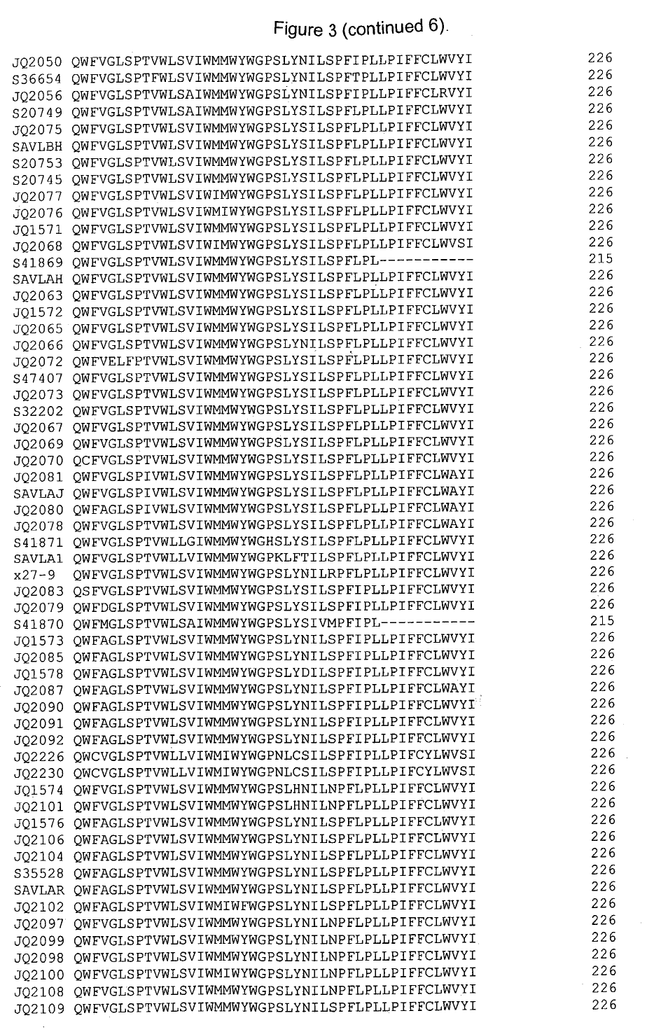

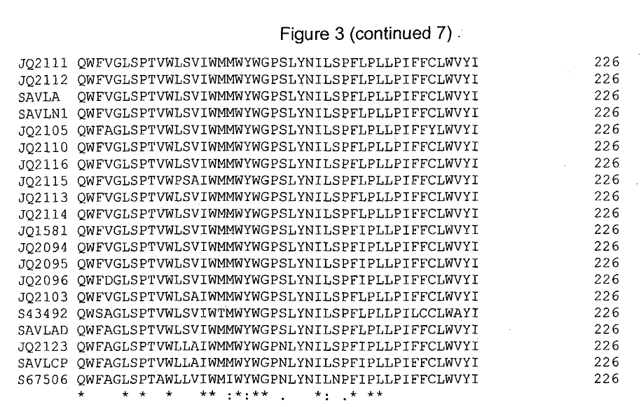

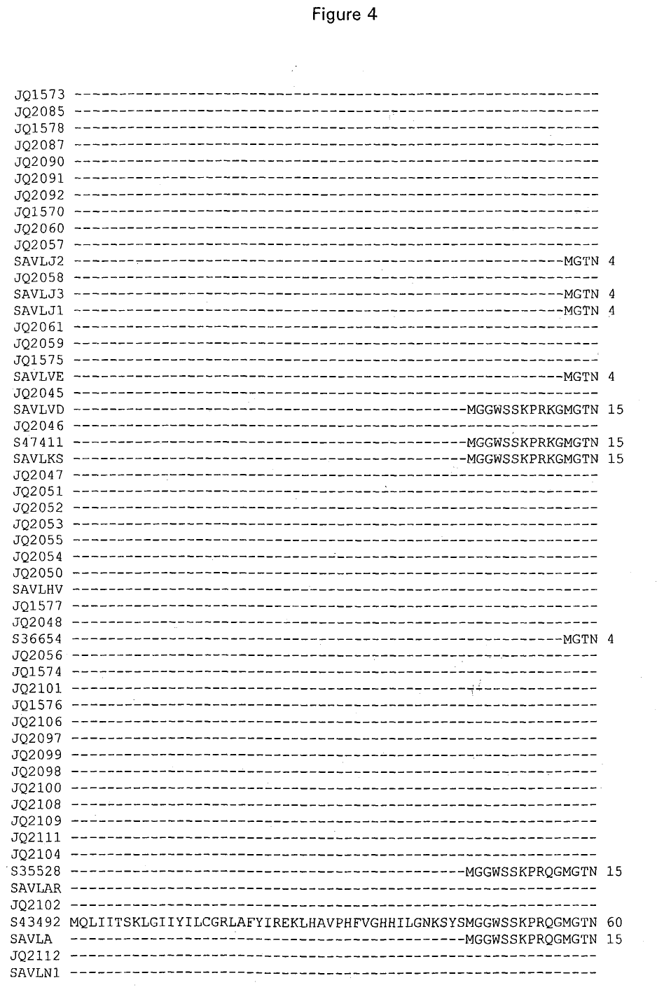

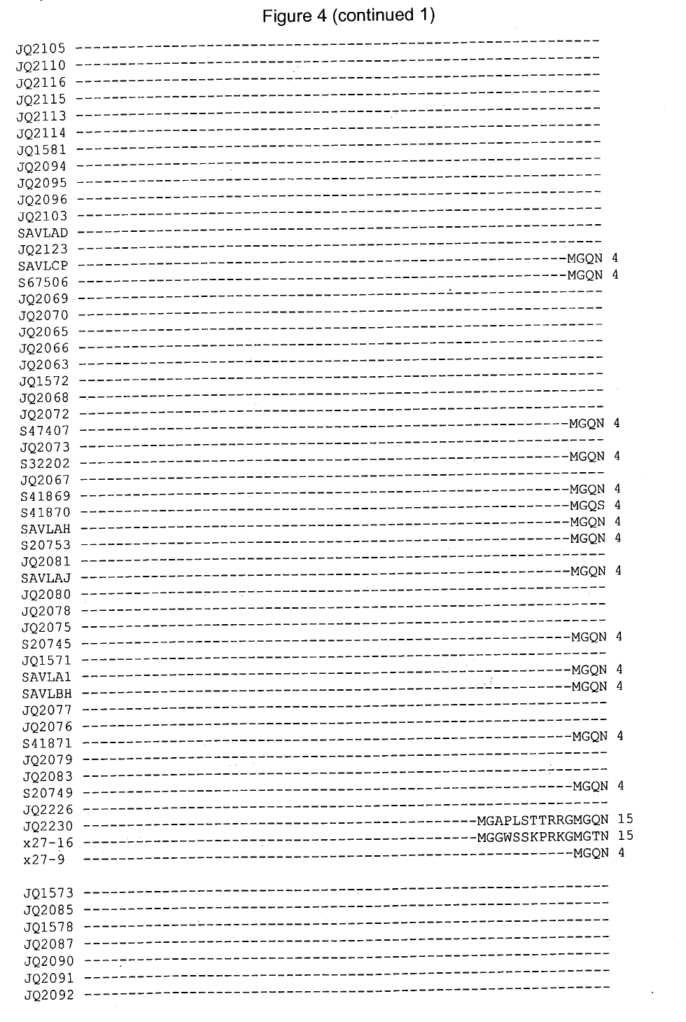

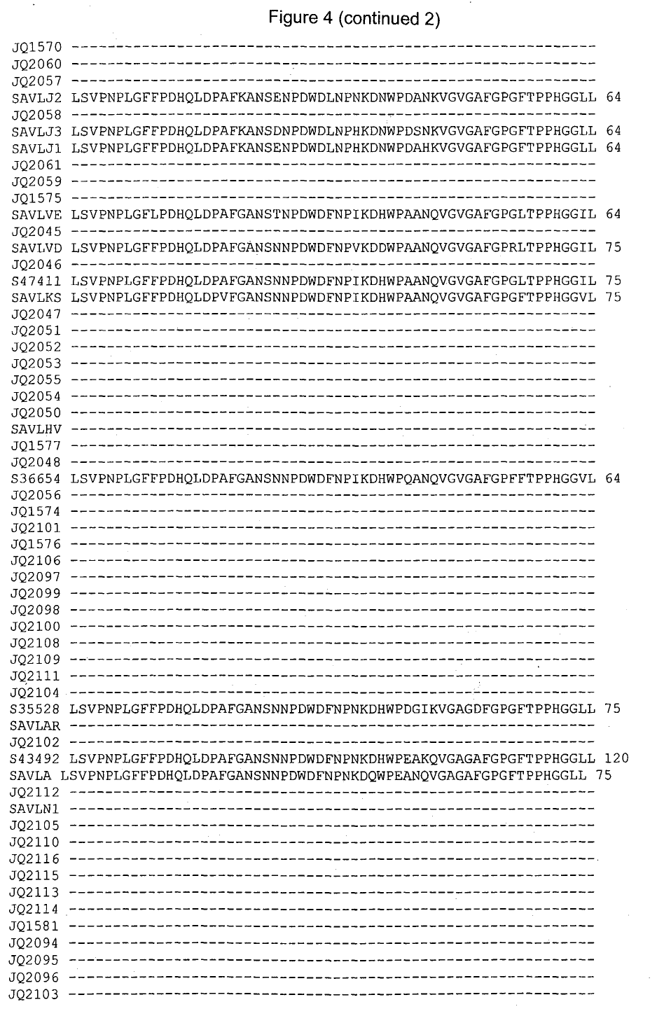

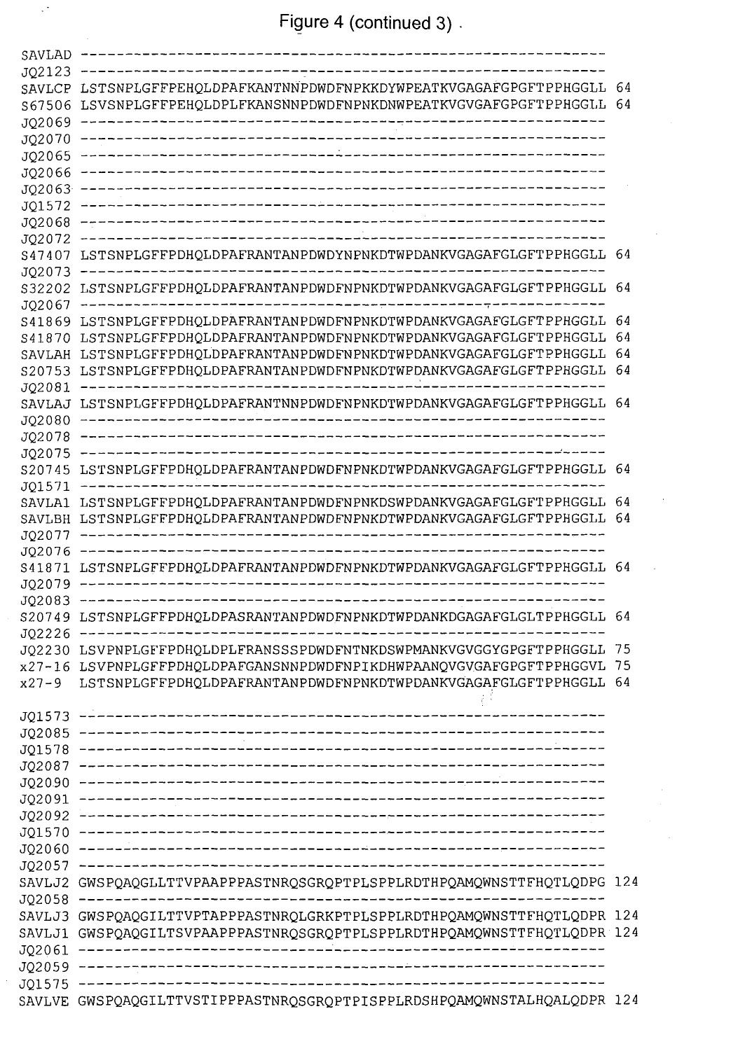

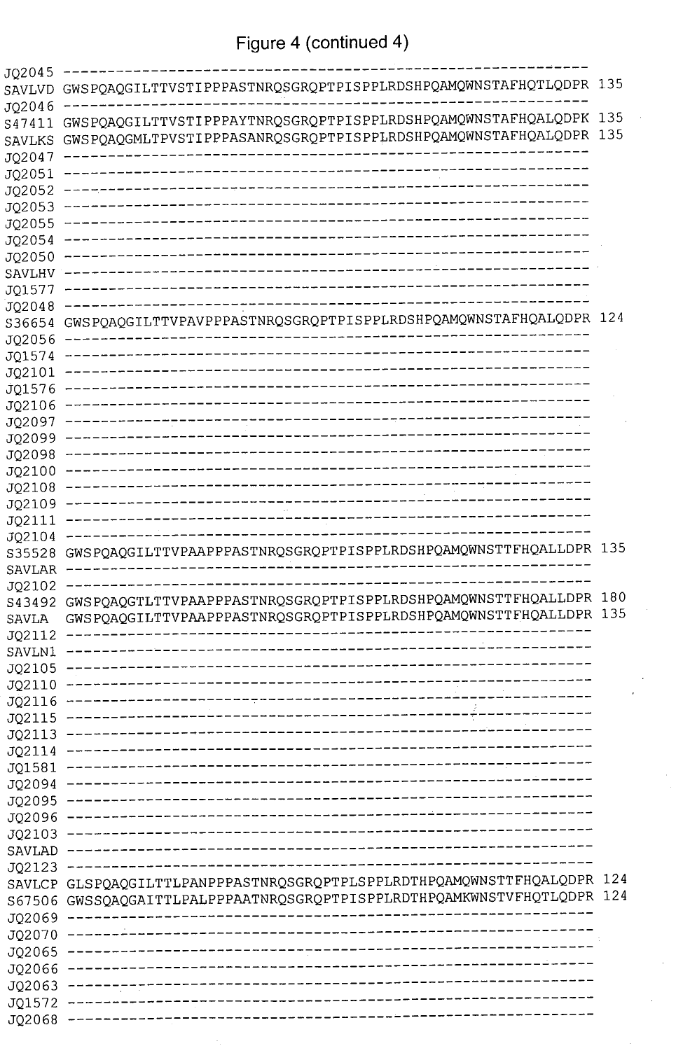

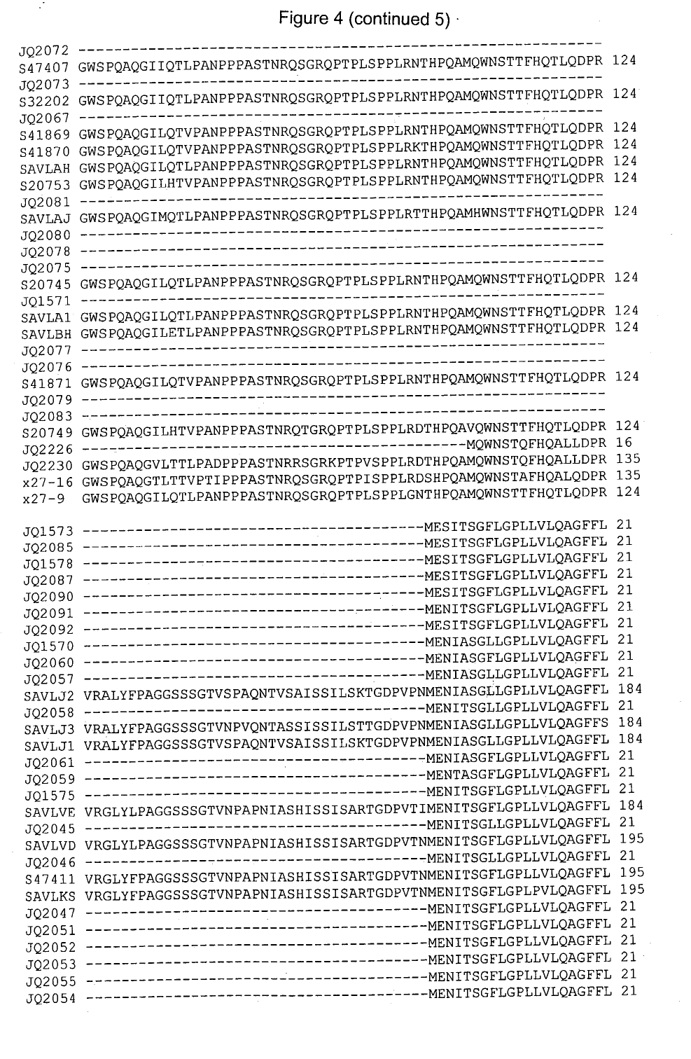

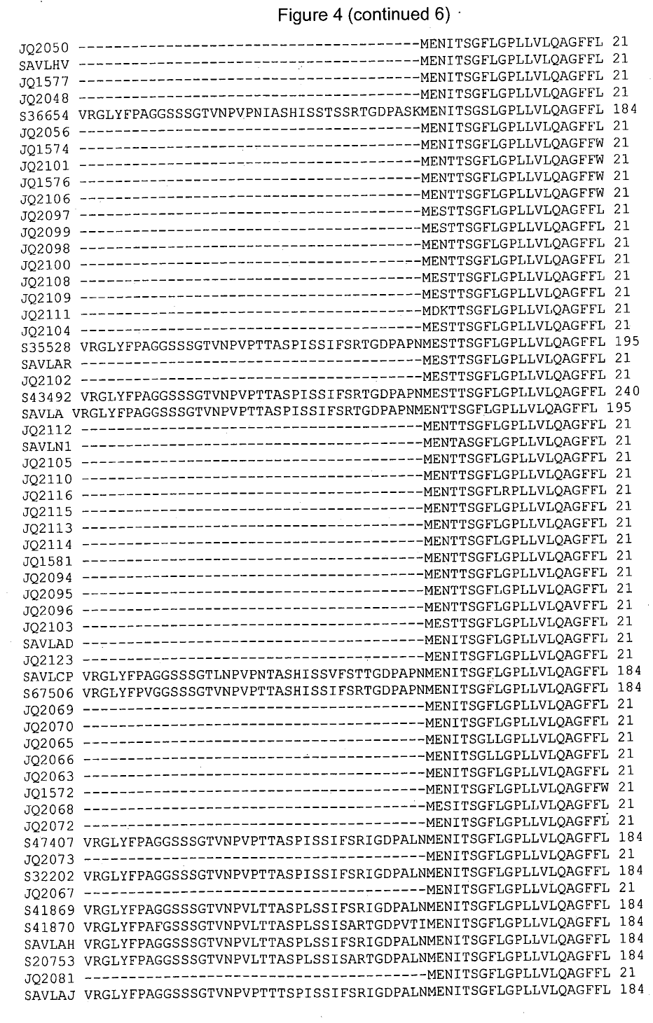

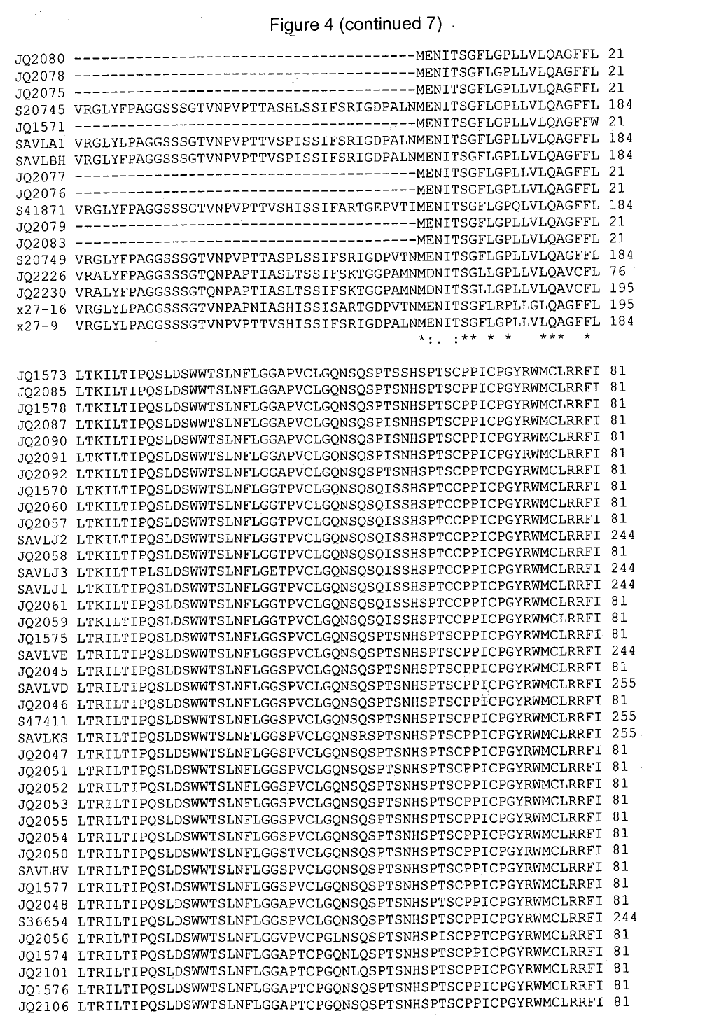

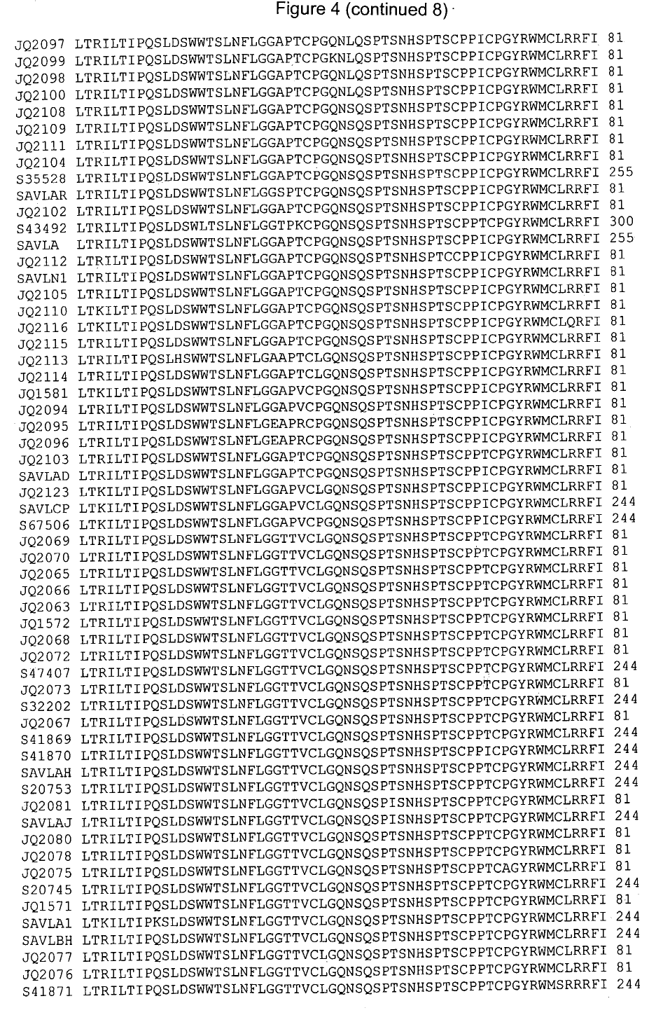

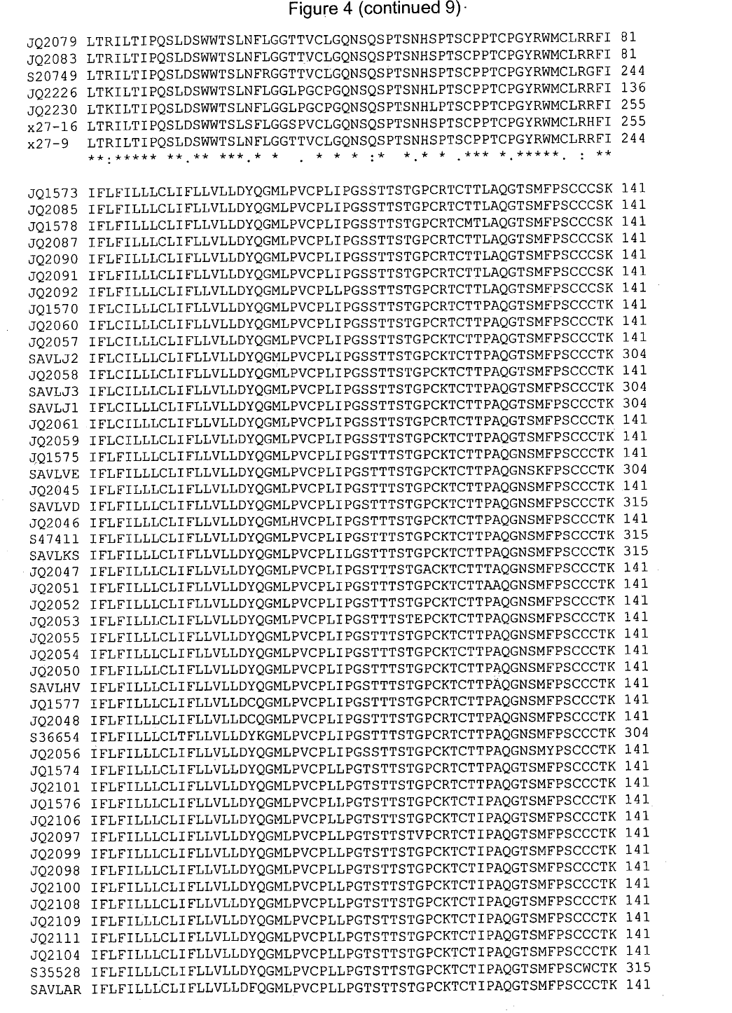

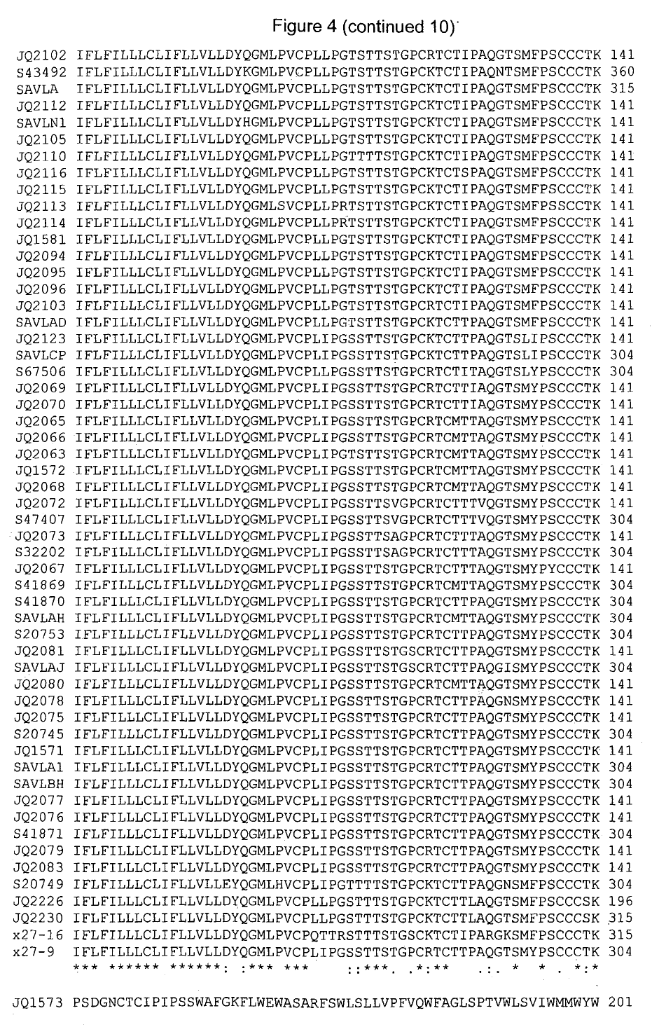

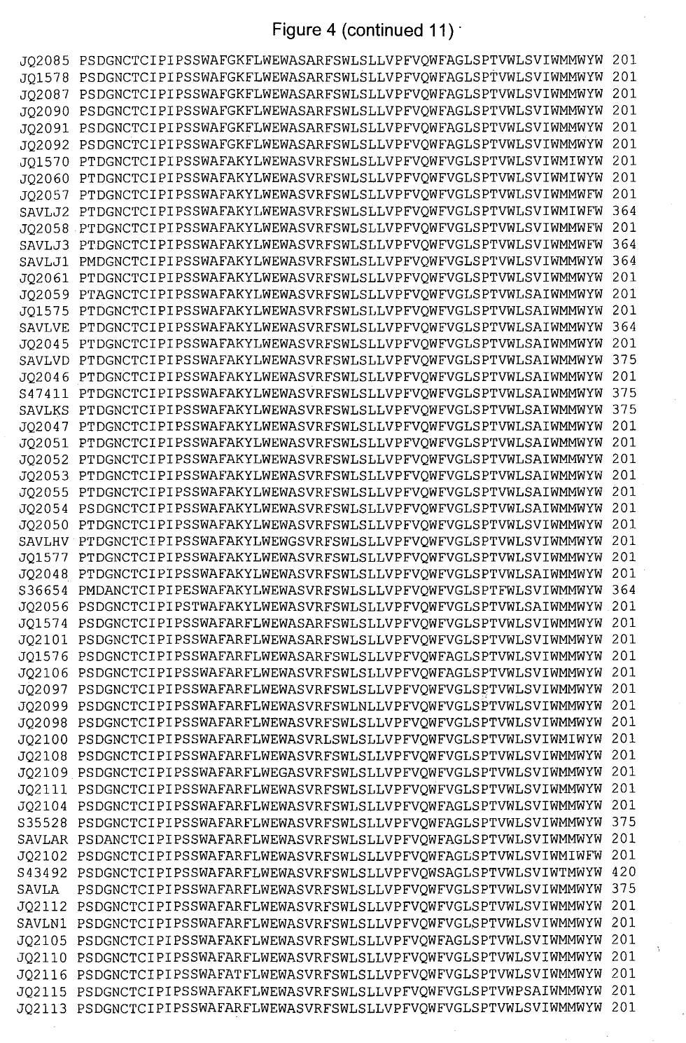

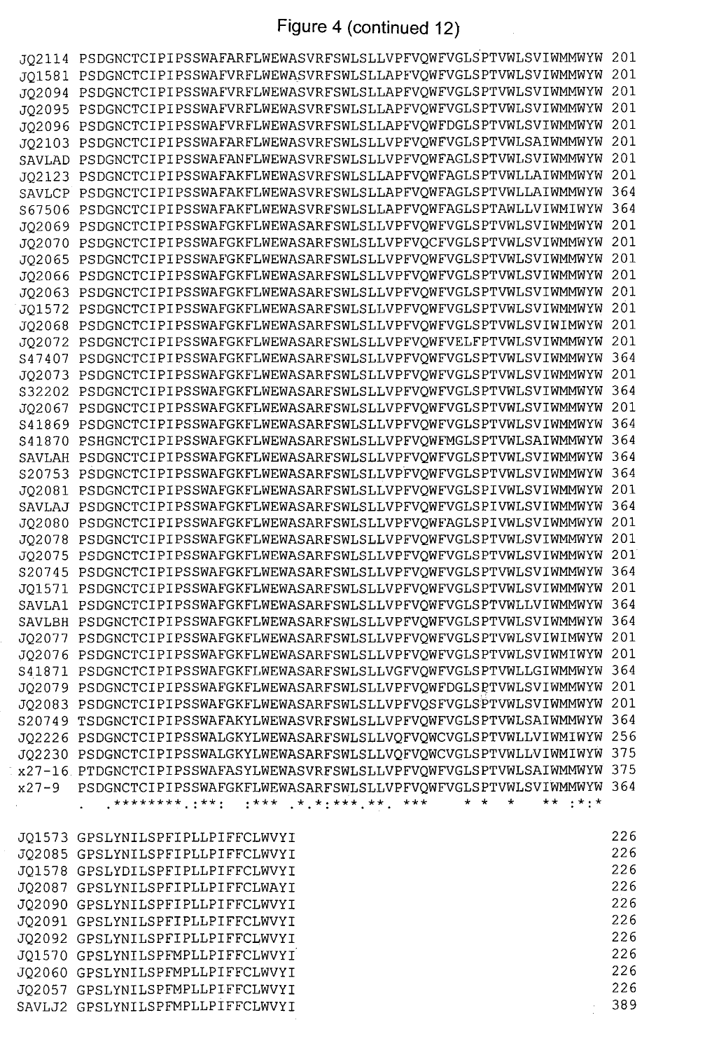

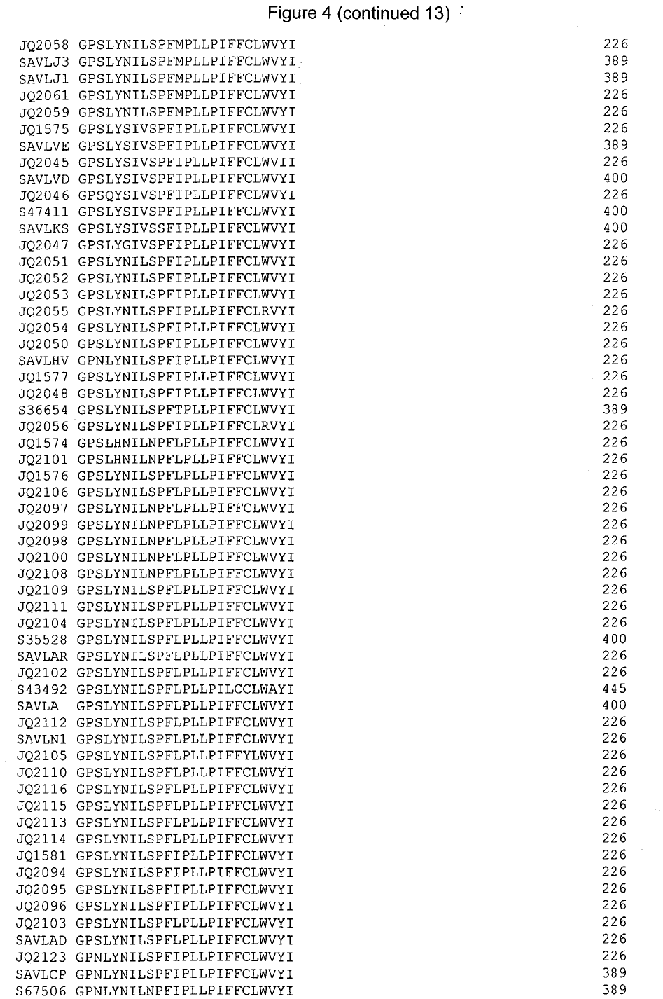

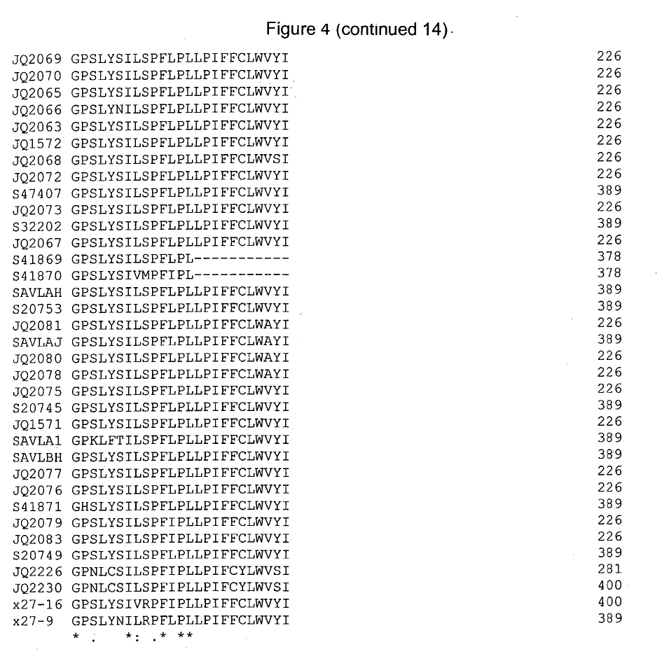

FIGS. 3 and 4 show, respectively, the amino acid sequences of the HBs gene and of the whole of the Pre-S region of the clones x27_{—}16 and x27_{—}9, aligned with 102 sequences HBs or Pre-S found in the NBRF/PIR bank.

### EXAMPLE 1

### Identification of the mHBV Variant or Mutant

The patient is a man of 86 years with a history of chronic non-A, non-B hepatitis, the pathology of which has developed to the stage of cirrhosis. He did not present any identified risk factor. His blood level of transaminases was appreciably higher than normal (1 to 2 times relative to the normal level). He was neither HCV, nor HGV, nor HDV positive in the tests for detecting RNA by PCR. Potential detection of the TTV virus was also effected by PCR and it proved negative.

1. Serological tests: detection of the HBsAg antigen was carried out in the patient's serum firstly with the second-generation MonoLisa test (trade name) marketed by Sanofi Diagnostics Pasteur and secondly with the VIDAS® HBsAg detection kit marketed by the company bioMérieux. The results were negative with each detection test employed. Detection of anti-HBs antibodies was carried out using the ELISA DiaSorin test (trade name) marketed by the Sorin company in the patient's serum. The results of the test for detecting anti-HBs antibodies were also negative. Detection of total anti-HBc antibodies was carried out by a CORAB rDNA (trade name) competitive test from the company Abbott Diagnostics and using the ELISA Ortho HBc Elisa test marketed by Ortho Diagnostic System. The results show the presence of 10⁴ DNA molecules per ml of serum in the patient compared with the 10⁸ DNA molecules/ml usually found in HBV-positive patients at a chronic stage of the disease.

2. Immunolabelling in the liver: immunolabelling was carried out on liver samples by immunofluorescence for the HBsAg antigen using polyclonal rabbit antibodies directed against HBsAg (marketed by the Janssen company) and fluorescent anti-rabbit antibodies (from the DAKO company). Labelling with peroxidase was effected using a polyclonal rabbit antibody directed against the HBc antigen and anti-rabbit antibodies (from DAKO) as described by Vitvitski-Trepo et al., Hepatology, 6: 1278-1283 (1990). These tests proved to be negative for the presence of the HBsAg and HBcAg antigens in the patient's liver.

3. Test for detecting the viral genome by PCR: detection of the viral genome in the serum by PCR was effected using the test Expand High Fidelity PCR System, marketed by the company Roche for HBV and using the AMPLICOR test (trade name) of the company Abbott for HCV. 140 μl of serum was used for the extraction of nucleic acids. The nucleic acid was extracted using a nucleic acid extraction kit marketed by the company Qiagen, which permits simultaneous purification of DNA and RNA. The serum was incubated in the presence of a lysis buffer for 10 minutes at room temperature, and was then passed through columns of silica. The DNA was eluted from the column with 50 μl of sterile water. The total content of nucleic acids from whole blood or from liver biopsies was extracted using the kits marketed by the company Qiagen, for the blood and tissues respectively.

The DNA of HBV was detected by nested amplification in two stages, by means of primers selected in a region that is known to be well conserved in the S gene of HBV.

First stage of amplification: the first 35 amplification cycles are preceded by preheating at 95° C. for 5 min, 95° C. for 45 seconds, 48° C. for 45 seconds and 72° C. for 1 min. An elongation stage for 10 minutes was then carried out.

Primer 1 (Pol 1): 5′ CCT GCT GGT GGC TCC AGT TC 3′ (Pichoud et al., Hepatology; 1999, 1: 230-237)

Primer 2 (POR4): 5′ TAC CCA AAG ACA AAA GAA AAT TGG 3′

The second stage of amplification is of 40 cycles with preheating at 95° C. for 5 min, 5 cycles of amplification at 95° C. for 25 seconds, 37° C. for 45 seconds, 72° C. for 1 minute and the other 35 cycles at 95° C. for 45 seconds, 48° C. for 45 seconds and 72° C. for 1 min. At the end, an elongation stage was carried out.

| | |
|---|---|
| Primer 3: | 5′ TAG TAA ACT GAG CCA RGA GAA AC 3′ |
| Primer 4: | 5′ GTT GAC AAR AAT CCT CAC AAT AC 3′ |

R represents A or G.

PCR is carried out starting from 10 μl of total nucleic acids that were extracted from 140 μl of serum, 200 μl of whole blood, and 25 mg from a liver biopsy frozen or fixed with formalin in paraffin.

PCR amplification of the X gene was also carried out following the technique described by Uchida et al., Microbiol. Immunol., 1994, 38, 281-285. Amplification of the X gene is interesting because the product of the X gene transactivates the HBV promoters and because the promoter of the core of HBV overlaps the X gene. Consequently, mutations affecting either the product of the X gene or the promoter of the core or both, could lead to a decrease in the levels of transcription and replication of the virus, explaining why it cannot be detected by the commercial tests.

1st round:

| | |
|---|---|
| primer 1: | 5′ CCA TAC TGC GGA ACT CCT AG 3′ |
| primer 2: | 5′ ATT TGC TCG CAG CCG GTC TG 3′ |

2nd round:

| | |
|---|---|
| primer 3: | 5′ TTT TGC CAG CCG GTC TG 3′ |
| primer 4: | 5′ ATT TGC TCG CAG CCG GTC TG 3′ |

Serial dilutions of plasmid diluted in a control serum enabled us to determine the sensitivity of the PCR. Nested PCR in the S and X genes of HBV makes it possible to detect ten genomes of HBV.

4. Quantification of the DNA of HBV in the serum: quantification was effected using the test Amplicor HBV monitor (trade name), marketed by the company ROCHE. The test comprises primers chosen in the pre-C/C region of HBV which make it possible to detect from 4×10² to 4×10⁷ copies of DNA of HBV/ml. 50 μl of serum was used in this test (Kessler et al., Clin. Chem., 1998; 36: 601-604).

5. Amplification of the complete genome of HBV: amplification of the HBV genome was carried out using the technique described by Günther et al., Journal of Virology, September 1995, pages 5437-5444 using the following primers:

Primer 1 (P1):
5′ CCG GAA AGC TTG AGC TCT TCT TTT TCA CCT CTG
CCT AAT CA 3′
Primer 2 (P2):
5′ CCC GAA AGC TTG AGC TCT TCA AAA AGT TGC ATG
GTG CTG G 3′

The kit Expand High Fidelity (trade name) marketed by the company Roche containing 1.5 mmol/l of MgCl₂, 200 μmol/l of deoxynucleoside triphosphate, 2.6 U of a mixture of Taq and Pwo DNA polymerase (High Fidelity, marketed by the company Roche) were used with 1 μmol/l of each primer described above.

The technique exploits the fact that although the HBV genome found in the circulating virions is circular, the strands are not closed covalently and can therefore hybridize rapidly with the PCR primers and the fact that there is a short terminal redundancy at the 5′ and 3′ ends of the minus strand. The 3′ end of primer P1 is complementary to the 5′ end of the minus strand, including the redundant sequence, whereas the 3′ end of primer P2 is complementary to the sequence of the plus strand, beginning with the redundant sequence. The 5′ ends of the two primers contain the sites of restriction enzymes for Hind III, Sac I and Sap I that are rarely found in the genomes of HBV.

After a warm start, 40 cycles of PCR were carried out with denaturing at 94° C. for 40 seconds, hybridization at 60° C. for 1.30 minutes and elongation at 72° C. for 3 min, with a 10-second increment after each cycle, in apparatus for PCR amplification marketed by the company Perkin Elmer. A final extension of 10 minutes is effected at the end of the cycles.

Production of the positive DNA strand by the action of viral polymerase was carried out as described by Hantz et al., Antimicrobiol. Agent Chemether, 1984, 25: 240-246, with incubation of the serum in the presence of DNTPs, at a temperature of 37° C. over night. When the genome was not amplified or was only slightly amplified, PCR aliquots of the whole genome were reamplified using a combination of primer P1 and primer Por1 or of primer P2 and primer Pol1. These semi-nested PCRs each make it possible to obtain a fragment of about 1800 base pairs, with overlaps from one to another of about 300 base pairs.

6. Analysis of the PCR products: aliquots of the products of the PCR reaction were subjected to electrophoresis on agarose gels (1 to 2% depending on the assumed size of the amplified product). The gels were stained with ethydium bromide or with the GELSTAR stain, and photographed. The DNA was then transferred to a nylon membrane (Hybon N+nylon membrane) marketed by Amersham, in the presence of 0.4 M of NaOH. The nylon membrane was then subjected to hybridization with a 3.2-kb DNA probe of HBV, labelled with ³²P using the “Ready to go” kit Random Primer Kit, marketed by the company Pharmacia Biotech.

7. Purification of the PCR products: when the specific bands of HBV have been amplified, the remainder of the PCR reaction products are subjected to agarose gel electrophoresis, stained, and the specific bands are excised using a scalpel under UV irradiation at wavelength of 312 nm. The DNA is then isolated from the agarose gel using the Geneclean kit, marketed by Bio 101, and eluted in a small volume of water.

8. Cloning of the products of PCR amplification: the cloning strategies used depend on the nature of the products of PCR amplification. The amplifications of subgenomic HBs and HBx were carried out with Taq polymerase which possesses an activity of the natural terminal transferase type and the addition of a nucleotide, usually adenosine, to the 3′ ends of the amplified fragments. These fragments are cloned directly in the pGEM-T vector (Promega), a linearized cloning vector containing an additional codon that codes for thymine at its 3′ end. The amplifications of the complete genome by PCR or the subsequent semi-nested PCR amplifications were carried out with a mixture of Taq and Pwo polymerases and the fragments amplified are blunt-ended fragments or are a mixture of molecules with blunt ends or an additional A residue at the 3′ end. The said fragments are either cloned in a blunt-ended vector, the pSTBLUe-1 vector, after conversion of all the fragments to blunt-ended fragments (Perfectly Blunt Cloning Kit, Novagen), or an A residue is added to the 3′ ends by incubating the fragments with Taq polymerase in the presence of DATP and then cloning them in the pGEM-T vector. After ligation, according to the instructions recommended by the manufacturer, the ligation products are purified using a PCR resin Preps resin, marketed by the company Promega, and eluted in water. Aliquots are used for transforming the cells of E. coli XL-2 Blue by electroporation. The transformed cells are spread on plates of LB agar containing ampicillin (100 μg/ml), IPTG (80 μL) and X-gal (70 μg/ml) and incubated over night at 37° C.

9. Identification of HBV recombinant plasmids: initial screening involves selecting white colonies that may contain the insert of interest, in contrast to the blue colonies that are assumed not to contain the insert. If the transformation seems to be particularly successful, i.e. if we observe a consistent quantity of white colonies relative to the blue colonies constituting the negative control, the white colonies are then amplified directly in a liquid medium (Terrific Broth containing 100 μg/ml ampicillin) over night at 37° C. with stirring. Otherwise, the white colonies are transferred to two LB agar plates containing ampicillin. One of the plates has a nylon membrane on the surface of the agar. About 100 colonies can be transferred to each plate. The plates are incubated over night at 37° C. The plate without the nylon membrane is stored at 4° C. The nylon membrane is treated with NaOH for lysis of the bacteria, then neutralized and dried. After fixing the DNA by UV irradiation, the membrane is subjected to hybridization with specific, ³²P-labelled HBV probes. The appropriate colonies are then taken from the main plate and amplified in a liquid medium.

10. Purification of the plasmid DNA: the DNA is purified by mini preps using Qtips-20, marketed by the company Qiagen, in accordance with the instructions recommended by the company. Other DNAs are prepared manually by alkaline lysis, precipitation of the supernatant with isopropanol, resuspension and treatment of the residue with phenol then chloroform and precipitation with ethanol. In both cases, the DNA is finally resuspended in 30 μl of TE. The quantities of DNA are estimated by agarose gel electrophoresis.

11. Sequencing: approximately 350 ng of plasmid is sequenced with 5 pmol of primers, using the BigDye sequencing kit marketed by the company Perkin Elmer and amplification equipment (9600 thermal cycler from Perkin Elmer). After purification of the sequencing products by spin chromatography on Sephadex G50, the sequencing products are analysed on an ABI Prism 377 sequencer, marketed by the company Applied Biosystems. Initial sequencing is usually carried out with primers that are complementary to the sequences of the T7 and SP6 promoters which overlap the cloning kit simultaneously in the pGEM-T and pSTBlue-1 vectors. For short fragments, of less than 500 to 600 base pairs, good information on the sequences of the two strands of the insert can usually be obtained with these two primers. For longer inserts, such as those of complete HBV genomes, HBV-specific primers are selected from the HBV genome, roughly at intervals of 500 base pairs, in both directions, so that the two strands can be sequenced completely. Several clones of each fragment are sequenced.

12. Sequence analysis: most of the data analyses were carried out using programs available on the server INFOBIOGEN (Villejuif, France). The data of the raw sequences are corrected using the program of the sequencer. At this stage, only the ambiguities (called N by the machine) and the obvious base errors near the ambiguities are corrected. Then a search by BLAST is carried out, in most cases, to identify the HBV sequence of the base that is closest to that of the clone of the invention. Complete alignment, using the CLUSTAL W software, of this sequence and of the sequence of the clone permits additional correction. Only obvious errors, such as the omission of bases by the machine or the addition of bases at the end of the sequence are corrected. The partially corrected sequences on the two strands of the clone are assembled and aligned. Any conflicts are resolved by choosing the sequence that is best supported by the data. Finally, the corrected sequences of the various clones from the same patient are compared and the differences are verified again. To facilitate biological analysis of the amplified sequences, 81 sequences of the complete genome of HBV found in the databases were aligned using CLUSTAL W. Phylogenic analysis of this alignment shows that the sequences of the invention belong to 6 main groups, corresponding to genotypes A to F, determined previously by sequence alignment of the HBs gene. On adding sequences derived from cryptic HBV genomes to this alignment, it is possible to ascribe these genomes to a particular genotype or establish that the cryptic HBV genomes belong to an as yet unknown genotype. Moreover, the amino acid sequences of the various proteins of HBV found in the databases (PreS/s, HBs, HBc, HBx, PreC/c and pol) were aligned. This permits comparison of the amino acid sequences deduced from the nucleotide sequences of the clones of the invention with the protein sequences of HBV already described, which are probably derived from non-cryptic HBV genomes. It was thus possible to identify mutations in the proteins of the cryptic HBV virus of the invention that are absent from the HBV isolates described before and are not due simply to a variation of the genotype.

13. Functional analysis: functional analysis was carried out with the clones derived from amplifications of the complete genome. The clones are cut with the restriction enzyme Sap I. The recognition and cutting sites of this enzyme are CTCTTCNNNN. This site is present in the primers P1 and P2 used for amplification of the whole genome. This results in release of an insert from the whole genome with cohesive ends and, because of the terminal redundancy of the minus strand, recircularization in a complete HBV genome without superfluous sequences. After cutting with the restriction enzyme Sap I, the DNA is extracted with phenol and precipitated with ethanol. The DNA is then transfected in HuH7 cells, a cell line of human hepatocarcinoma permissive for the replication of HBV, using the transfection reagent FuGene-6, marketed by the company Roche. Recircularization of the insert takes place in the cells and permits transcription and viral replication. The negative control consists of cells transformed with a pGEM vector without insert and the positive controls are a dimer of the complete genomes of a wild-type HBV cloned at the EcoRi site, intact or cut with the EcoRi restriction enzyme. Transfection is controlled by co-transfection with pSEAP, a plasmid expressing Soluble Excreted Alkaline Phosphatase, the activity of which is measured in the culture medium 48 hours after transfection. The media are changed daily and the media from days 2 to the end of the experiment (normally day 5 or 6) are collected. The presence of the HBs antigen in the medium is measured using the Ausria kit, marketed by the company Abbott. At the end of the experiment, the cells are lysed and the DNA, the RNA and the proteins are extracted. The medium is stored and concentrated by precipitation with PEG. The presence of viral proteins, DNA or RNA is analysed in the various preparations. For the sub-genomic clones, the fragments are inserted in HBV expression vectors, either in the context of a complete genome for studying the effects of mutations of the HBs gene, of the HBx gene or of the core promoter on the replication cycle, or in expression vectors for HBs or HBx for studying the properties of the mutated proteins, in particular to find out whether the mutated HBs proteins are synthesized and secreted normally and whether they are recognized by the commercial detection tests.

14. Results: according to the protocols described above, the complete HBV genome of the patient was amplified by PCR and cloned. Two PCR amplifications were carried out and independent clonings were effected. Phylogenic analysis shows that the genome is of genotype A, but it is on the borderline of the family and might represent a new subgroup in genotype A. The important elements of structure and regulation are conserved in the nucleotide sequences and there are no major changes in the amino acid sequences deduced from HBx and from pol relative to the wild type. The genome is therefore competent for its replication. However, the amino acid sequence and the HBsAg protein have numerous substitutions, especially in the a determinant, which corresponds to a part of the antigen exposed on the surface of the viral particles and is recognized by the commercially available serological tests. It is particularly interesting to note that there are successive substitutions in positions 109-112 of the amino acid sequence of the HBsAgm protein and other substitutions isolated at the level of HBsAgm. The main substitutions are in HBsAgm QTTR (amino acids 109 to 112) instead of LIPG found at the level of the protein of wild-type HBsAg. These substitutions, among other things, can induce a change in antigenicity of the HBsAgm protein and can explain why it is not recognized by the tests available commercially. Moreover, a mutation is also found at the level of the Pre-Sm protein that consists of replacement of the amino acid Ile found at position 84 of the wild-type protein, referenced at SEQ ID NO 4, by the amino acid Thr in the Pre-Sm protein (position 84 of SEQ ID NO 4).

### EXAMPLE 2

### Identification of an m′HBV Mutant or Variant

In the same patient of 86 years with a history of chronic non-A, non-B hepatitis, another mutated HBV virus was also identified (m′HBV). The genome of this virus was amplified, cloned and sequenced as described in Example 1. Phylogenic analysis shows that its genome is of genotype D. Sequence analysis shows that m′HBV is a pre-core mutant with a stop codon in PreC/c, just before the HBc gene. Furthermore, it has a mutation in the Pre-S part, just with the start of HBs, which consists of replacement of an Arg by a Gly in position 102 of the Pre-Sm′ protein identified in SEQ ID NO 6 and a mutation near the end of HBs, also shared with HBsm that consists of replacement of a serine by an arginine in position 210 of SEQ ID NO 5. For the purposes of simplification the nucleotide sequences coding for SEQ ID NO 6 and SEQ ID NO 5 have not been included in the present description, but the inventors have carried out a complete sequencing that makes it possible to assert that Gly is encoded by the GGA codon in position 304-306 of the Pre-S region or in position 3151-3153 relative to the sequence of the complete genome and that Arg is encoded by the AGG codon, in position 628-630 of the S gene or in position 782-784 relative to the sequence of the complete genome.

1. An isolated mutated HBV virus (mHBV) having the following characteristics:
(i) a genome with partially double-stranded, circular DNA,
(ii) the said genome including the genes Pre-S, S, C, P and X,
(iii) the Pre-S gene coding for surface antigens, the S gene coding for an envelope protein HBsAg, the C gene coding for a protein HBeAg and a protein HBcAg, the P gene coding for a DNA polymerase/reverse transcriptase enzyme and the X gene coding for a protein HBxAg, characterized in that the S gene contains a DNA nucleotide sequence referenced SEQ ID NO 1 and in that the Pre-S gene contains a DNA nucleotide sequence referenced SEQ ID NO 3.

2. A modified surface protein, characterized in that it consists of a peptide sequence chosen from SEQ ID NO 2 and SEQ ID NO 4.

3. A DNA or RNA nucleotide fragment, characterized in that it includes a DNA or RNA nucleotide sequence of at least 12 nucleotides, preferably of at least 15 or 18 nucleotides and advantageously of at least 21 nucleotides that includes the nucleotides 325 to 336 of SEQ ID NO 1 and/or the nucleotides 235 to 237 of SEQ ID NO 1 and/or the nucleotides 391 to 393 of SEQ ID NO 1 and/or the nucleotides 478 to 480 of SEQ ID NO 1 and/or the nucleotides 28 to 30 of SEQ ID NO 1 and/or the nucleotides 39 to 41 of SEQ ID NO 1 and/or the nucleotides 358 to 360 of SEQ ID NO 1 and/or the nucleotides 385 to 387 of SEQ ID NO 1 and/or the nucleotides 118 to 120 of SEQ ID NO 1 and/or the nucleotides 628 to 630 of SEQ ID NO 1, and/or a fragment that includes a sequence of at least 21 nucleotides comprising the nucleotides 250 to 252 of SEQ ID NO 3, or is the product of transcription of the said DNA nucleotide sequences, provided that when the fragment includes the nucleotides 628 to 630 of SEQ ID NO 1, the said fragment then also includes the nucleotides 325 to 336, and/or 235 to 237, and/or 391 to 393, and/or 478 to 480, and/or 28 to 30, and/or 39 to 41, and/or 358 to 360, and/or 385 to 387, and/or 118 to 120 of SEQ ID NO 1, and/or the nucleotides 250 to 252 of SEQ ID NO 3.

4. A DNA or RNA nucleotide fragment, characterized in that it consists of a nucleotide sequence which corresponds to the DNA nucleotide sequences SEQ ID NO 1 and SEQ ID NO 3 or the complementary sequences of the said sequences SEQ ID NO 1 and SEQ ID NO 3 or the RNA nucleotide sequences that are the products of transcription of sequences SEQ ID NO 1 and SEQ ID NO 3.

5. A DNA or RNA nucleotide fragment, characterized in that it consists of a DNA nucleotide sequence that corresponds to SEQ ID NO 1 and SEQ ID NO 3 or the complementary sequences of the said sequences SEQ ID NO 1 and SEQ ID NO 3 or in that it consists of an RNA nucleotide sequence that corresponds to the products of transcription of sequences SEQ ID NO 1 and SEQ ID NO 3.

6. A DNA molecule, characterized in that it includes a DNA nucleotide sequence chosen from SEQ ID NO 1, SEQ ID NO 3, their fragments according to one of the claims 3 to 5, and their complementary sequences.

7. An RNA molecule, characterized in that it includes an RNA nucleotide sequence that is the product of transcription of a DNA nucleotide sequence chosen from SEQ ID NO 1, SEQ ID NO 3, their fragments according to one of the claims 3 to 5, and their complementary sequences.

8. A protein fragment, characterized in that it includes a peptide sequence of at least 4 amino acids, preferably of at least 5 or 6 amino acids and advantageously of at least 7 amino acids, especially from 6 to 15 amino acids and advantageously from 6 to 10 or from 8 to 12 amino acids and that includes the amino acids 109-112 and/or 79 and/or 131 and/or 160 and/or 10 and/or 14 and/or 120 and/or 129 and/or 40 and/or 210 of SEQ ID NO 2, and/or a fragment that includes a peptide sequence of at least 7 amino acids including amino acid 84 of SEQ ID NO 4, provided that when the fragment includes amino acid 210 of SEQ ID NO 2, the said fragment then also includes the amino acids 109-112 and/or 79 and/or 131 and/or 160 and/or 10 and/or 14 and/or 120 and/or 129 and/or 40 of SEQ ID NO 2 and/or amino acid 84 of SEQ ID NO 4.

9. A protein fragment according to claim 8, characterized in that it consists of a peptide sequence that includes the peptide sequences SEQ ID NO 2 and SEQ ID NO 4.

10. A fragment according to claim 8, characterized in that it consists of a peptide sequence chosen from SEQ ID NO 2 and SEQ ID NO 4.

11. A protein fragment according to claim 8, characterized in that it consists of SEQ ID NO 2 and SEQ ID NO 4.

12. A modified surface protein, characterized in that it includes a peptide sequence chosen from SEQ ID NO 2, SEQ ID NO 4 and their fragments according to one of the claims 8 to 11.

13. A functional expression cassette in a cell originating from a prokaryotic or eukaryotic organism permitting expression of a DNA fragment as defined in claims 3 to 5, placed under the control of the elements necessary for its expression.

14. An expression cassette according to claim 13, characterized in that it is functional in a cell originating from a eukaryotic or lower eukaryotic organism.

15. An expression cassette according to claim 14, characterized in that the cell originating from a eukaryotic organism is selected from the COS and CHO cells and in that the cell originating from a lower eukaryotic organism is selected from the cells of Saccharomyces cerevisiae and of Pichia pastoris.

16. A vector containing an expression cassette according to one of the claims 13 to 15.

17. A cell originating from a prokaryotic or eukaryotic organism, preferably a eukaryotic or lower eukaryotic organism and advantageously a COS or CHO cell or a cell originating from Saccharomyces cerevisiae or from Pichia pastoris containing an expression cassette according to one of the claims 13 to 15 or a vector according to claim 16.

18. A surface protein produced by an expression cassette according to one of the claims 13 to 15, a vector according to claim 16 or a cell according to claim 17.

19. A method for preparing a modified surface protein according to one of the claims 4 or 5 or a protein fragment according to claims 9 to 12 that consists of culturing a host cell according to claim 17 in a suitable culture medium, the said host cell being transformed with an expression vector that contains a DNA nucleotide sequence as defined in claim 6 or a nucleotide fragment as defined in claims 3 to 5 and of purifying the said modified surface protein produced to a required degree of purity.

20. An immunogenic peptide, characterized in that it has a peptide sequence as defined in claims 2, and 8 to 12 or in that it consists of a protein as defined in claims 18 and 19.

21. A monoclonal antibody, characterized in that it is obtained by immunization of a mammal with an immunogenic peptide as defined in claim 20, in that it is specific to the modified surface protein defined in claim 12, and in that it does not recognise the wild-type protein.

22. Polyclonal antibody, characterized in that it is obtained by immunization of a mammal with an immunogenic peptide as defined in claim 20, in that it does not recognise the wild-type protein.

23. A diagnostic composition, characterized in that it consists of a protein or a protein fragment as defined in claims 2, 8 to 12, 18 and 19.

24. A method for detecting antibodies directed against at least one mutated surface protein that consists of SEQ ID NO 2 and/or SEQ ID NO 4 in a biological sample, according to which the biological sample is placed in contact with a diagnostic composition as defined in claim 23 under predetermined conditions permitting the formation of antibody/antigen complexes and the formation of the said complexes is detected.

25. A diagnostic composition, characterized in that it consists of a monoclonal antibody or a polyclonal antibody as defined in claim 21 or 22.

26. A method for detecting one mutated surface protein that consists of SEQ ID NO 2 and/or SEQ ID NO 4 in a biological sample, according to which the biological sample is placed in contact with a diagnostic composition as defined in claim 25 under predetermined conditions permitting the formation of antibody/antigen complexes and the formation of the said complexes is detected.

27. Biological material for the preparation of a pharmaceutical composition intended for treating human beings infected with at least the mHBV virus, the said composition comprising:
(i) either at least one natural protein and/or recombinant protein and/or synthetic polypeptide or their fragments whose sequence corresponds to all or part of the sequence identified in SEQ ID NO 2 and/or of the sequence identified in SEQ ID NO 4, optionally combined with at least one natural protein and/or recombinant protein and/or synthetic polypeptide or their fragments whose sequence corresponds to all or part of the sequence identified in SEQ ID NO 5 and/or of the sequence identified in SEQ ID NO 6 and/or at least one natural protein and/or recombinant protein and/or synthetic polypeptide or their fragments of a wild-type HBs antigen and/or of a wild-type Pre-S protein; or
(ii) at least one monoclonal or polyclonal antibody or fragment of the said antibodies, specific to at least one of the proteins referenced SEQ ID NO 2 and 4 or their fragments, optionally associated with at least one monoclonal or polyclonal antibody or fragment of the said antibodies specific to at least one of the proteins referenced SEQ ID NO 5 and SEQ ID NO 6 and/or with at least one monoclonal or polyclonal antibody or fragment of the said antibodies specific to at least one wild-type HBs or Pre-S protein.

28. An immunogenic or vaccinal composition, characterized in that it consists of a protein or a protein fragment as defined in claims 2, 8 to 12, 18 and 19, optionally combined with a suitable vehicle and/or adjuvant and/or diluent and with a pharmaceutically acceptable excipient.

29. A pharmaceutical composition, characterized in that it includes a biological material as defined in claim 27, optionally combined with a suitable vehicle and/or adjuvant and/or diluent and with a pharmaceutically acceptable excipient.

30. A probe, characterized in that it is capable of hybridizing under defined stringency conditions with a DNA or RNA nucleotide sequence as defined in claims 6 and 7 or with a nucleotide fragment as defined in claims 3 to 5.

31. A primer, characterized in that it is capable of hybridizing under defined stringency conditions with a DNA or RNA nucleotide sequence as defined in claims 6 and 7 or with a nucleotide fragment as defined in claims 3 to 5.

32. An anti-nucleic acid antibody, characterized in that it is capable of binding to a DNA or RNA nucleotide sequence as defined in claims 6 and 7 or with a nucleotide fragment as defined in claims 3 to 5.

33. A diagnostic composition, characterized in that it consists of one probe or one primer or one anti-nucleic acid antibody as defined in claims 30, 31 and 32.

34. A diagnosis method for viral DNA and/or RNA, according to which a sample of serum or plasma is taken from a patient, the said sample is treated if necessary to extract its DNA and/or RNA, the said sample is placed in contact with one probe or one primer as defined in claims 30 and 31, under defined stringency conditions, and the presence of viral DNA and/or RNA in the sample is detected either by demonstrating hybridization of the said viral DNA and/or RNA with a probe, or by amplification of the said DNA and/or RNA.

35. A diagnosis method for viral DNA and/or RNA, according to which a sample of serum or plasma is taken from a patient, the said sample is treated if necessary to extract its DNA and/or RNA, the said sample is placed in contact with one anti-nucleic acid antibody as defined in claim 32, the said antibody optionally being labelled with any suitable marker, and the formation of a nucleic acid/antibody complex is demonstrated.

36. A vaccinal composition consisting of a DNA sequence coding for one mutated surface protein of the mutated HBV virus (mHBsAg) as defined in claim 12 or for its fragments as defined in one of claims 8 to 11, the said protein mHBsAg including a modified determinant a of a protein HBs shown in SEQ ID NO 2, the said DNA being mixed with a suitable vehicle or diluent.

37. A composition according to claim 36, characterized in that in addition it contains a DNA sequence coding for at least one mutated surface protein shown in SEQ ID NO 4 or its fragments and/or a DNA sequence coding for at least one surface protein or its fragments shown in SEQ ID NO 5 and/or SEQ ID NO 6 and/or a DNA sequence coding for at least one wild-type HBs antigen and/or a wild-type Pre-S region.

38. An antisense or antigenic oligonucleotide, characterized in that it is capable of interfering specifically with the synthesis of a protein chosen from the proteins identified in SEQ ID NO 2 and/or SEQ ID NO 4 and/or SEQ ID NO 5 and/or SEQ ID NO 6.

39. A pharmaceutical composition, characterized in that it consists of an antisense oligonucleotide or an antigenic oligonucleotide as defined in claim 38.

40. A vector, characterized in that it includes at least one gene of therapeutic or vaccinal interest, the said gene coding in particular for:
(i) either at least one protein or protein fragment chosen from the proteins identified in SEQ ID NO 2 and/or SEQ ID NO 4 and/or SEQ ID NO 5 and/or SEQ ID NO 6;
(ii) or at least all or part of a polyclonal or monoclonal antibody capable of attaching to at least one of the proteins defined in (i) or to its fragments;
(iii) or at least one molecule that inhibits at least one of the proteins defined in (i);
(iv) or at least one ligand or any part of a ligand capable of attaching to at least one of the proteins defined in (i) or to a fragment of the said proteins and/or of inhibiting its function.

41. A therapeutic or vaccinal composition, characterized in that it contains, inter alia, a vector as defined in claim 40 and in that the said gene of interest is made dependent on elements ensuring its expression in vivo.

42. Biological material for the preparation of a pharmaceutical or vaccinal composition, containing at least one cell, especially a cell that does not produce antibodies naturally, in a form permitting its administration in a mammalian, human or animal, organism, as well as its optional prior culture, the said cell being genetically modified in vitro with at least one nucleic acid sequence containing at least one gene coding in vivo for at least one protein or protein fragment chosen from the proteins identified in SEQ ID NO 2 and/or SEQ ID NO 4 and/or SEQ ID NO 5 and/or SEQ ID NO 6 or coding for at least one molecule that inhibits the function and/or fixation and/or expression of at least one protein or protein fragment chosen from the proteins identified in SEQ ID NO 2 and/or SEQ ID NO 4 and/or SEQ ID NO 5 and/or SEQ ID NO 6 or coding for at least one antibody or antibody fragment capable of binding to at least one protein or protein fragment chosen from the proteins identified in SEQ ID NO 2 and/or SEQ ID NO 4 and/or SEQ ID NO 5 and/or SEQ ID NO 6.

43. A genetically modified cell, chosen in particular from the eukaryotic cells, such as the COS and CHO cells and the lower eukaryotic cells, such as yeast cells, in particular cells obtained from Saccharomyces cerevisiae and from Pichia pastoris, transformed by one nucleotide sequence or nucleotide fragment as defined in claims 6, 7 and 3 to 5 or by a vector as defined in claim 40.

44. A pharmaceutical or vaccinal composition, characterized in that it consists of a cell as defined in claims 42 and 43.

45. A method for evaluating a therapeutic agent according to which an animal is administered defined doses, in one dose or in repeated doses and at specified intervals of time, of at least one of the natural, recombinant or synthetic proteins or their fragments, or also obtained from plasma or serum, the said proteins being identified in SEQ ID NO 2 and/or SEQ ID NO 4, preferably SEQ ID NO 2 and SEQ ID NO 4 and optionally at least one of the natural, recombinant or synthetic proteins or their fragments, or also obtained from plasma or serum, the said proteins being identified in SEQ ID NO 5 and/or SEQ ID NO 6, preferably SEQ ID NO 5 and SEQ ID NO 6 and/or at least one of the natural, recombinant or synthetic proteins or their fragments, or also obtained from plasma or serum corresponding to a wild-type HBs antigen and/or to the wild-type Pre-S protein, preferably the wild-type HBs antigen and the wild-type Pre-S protein, a biological sample is taken from the animal, preferably from the blood or the serum and the following are carried out:
(i) assay of antibody or antibodies specific to the said protein or proteins; and/or
(ii) assay of the cellular immune response induced against the said protein or proteins or their fragments, for example by a test of activation in vitro of helper T lymphocytes specific to the said protein or proteins.
